(19) United States
(12) Patent Application Publication
(10) Pub. No.: US 2012/0065166 A1
(43) Pub. Date: Mar. 15, 2012

(54) Bone Modulators And Methods Therewith

(76) Inventors: Virginia Espina, Rockville, MD (US); Lance Liotta, Bethesda, MD (US); Antonella Chiechi, Matera (IT); Alessandra Romano, Catania (IT)

(21) Appl. No.: 13/209,742

(22) Filed: Aug. 15, 2011

Related U.S. Application Data
(63) Continuation-in-part of application No. 13/073,989, filed on Mar. 28, 2011.
Application No. 13/073,989 is a continuation-in-part of application No. PCT/US2009/004608, filed on Aug. 12, 2009.
(60) Provisional application No. 61/373,576, filed on Aug. 13, 2010.
(60) Provisional application No. 61/419,517, filed on Dec. 3, 2010.
(60) Provisional application No. 61/318,074, filed on Mar. 26, 2010.

(51) Int. Cl. A61K 31/47; A61K 31/506; A61K 31/517; A61P 19/08; A61K 31/663; C40B 30/04; G01N 33/53; G01N 33/535; A61K 31/4353; A61K 31/66

(52) U.S. Cl. 514/76; 514/313; 514/291; 514/252.19; 514/266.24; 514/108; 506/9; 435/7.8; 435/7.92

(57) ABSTRACT

THE INVENTION RELATES TO compositions, compounds, proteins and methods of treatment therewith. Aspects of embodiments also relate to a method of treating a patient by delivering to the tissues of said patient or administering to said patient a therapeutically effective amount of one or more compounds. Aspects of embodiments also relate to a method of detecting the presence of bone disease by machine-assaying detectable serotonin. Aspects of embodiments also relate to methods of treating disease in subjects.

### CROSS-REFERENCE TO RELATED APPLICATIONS

This application claims the benefit of U.S. Provisional Application No. 61/373,576, filed 13 Aug. 2010, and U.S. Provisional Application No. 61/419,517, filed 3 Dec. 2010, each of which is hereby incorporated by reference in its entirety. This application is also a continuation-in-part of U.S. application No. 13/073,989, filed 28 May 2011, which is a continuation-in-part (CIP) of PCT application PCT/US2009/004608, with an international filing date of 12 Aug. 2009, each of which is hereby incorporated by reference in its entirety. U.S. application No. 13/073,989, filed 28 May 2011 claims the benefit of U.S. Provisional Application No. 61/318,074, filed 26 Mar. 2010, each of which is hereby incorporated by reference in its entirety.

### INTRODUCTION

Bone disease is a major cause of morbidity in Multiple Myeloma (MM). Patients with bone disease may suffer from anemia, infections, bleeding, bony fractures of the vertebral column, osteolytic bone lesions, and impaired integrity of the skeleton. Skeletal integrity requires the harmonious activity of bone forming (osteoblasts) and bone-resorbing cells (osteoclasts). Multiple Myeloma-related bone disease may follow from a nonharmonic and uncoupled osteoblast inactivation and osteoclast activation adjacent to tumor foci within the bone. These and other challenges are addressed, and novel treatments for bone diseases are offered.

### BRIEF DESCRIPTION OF THE SEVERAL VIEWS OF THE DRAWINGS

These and other features, aspects, embodiments, and advantages of the present invention will become better understood with regard to the following description, appended claims, and accompanying drawings.

FIG. 1 is an artist's rendering of a non-limiting physiological mechanism of Serotonin-based regulation of bone mass as per aspect of an embodiment of the present invention. (Ducy et al. 2010).

FIG. 2 depicts three graphs illustrating a relationship between Serotonin, the angiogenesis switch (HIF-1α), and bone lesions in patients treated with bone breakdown inhibitors (bisphosphonates) as per aspect of an embodiment of the present invention.

FIG. 3 shows two graphs depicting the serum-Serotonin concentration and the platelet grains-Serotonin concentration for healthy, diseased, and disease-free bone tissue as per aspect of an embodiment of the present invention.

FIG. 4(A-M) is a multipanel figure depicting a correlation between Serotonin levels and RANK, DKK1, cytokines, and proteins that are known to regulate bone as per aspect of an embodiment of the present invention.

FIG. 5 depicts a two panel graph showing Serotonin's positive correlation with both IL-10 and TNFα as per aspect of an embodiment of the present invention.

FIG. 6 is a graph comparing the Serotonin, Rank, LRP6, Progesterone Receptor S190, Beta Arrestin, and DEPTOR concentrations in diseased and healthy bone as per aspect of an embodiment of the present invention.

FIG. 7 depicts two graphs comparing the response of aliquots of a single patient's bone marrow aspirate to (Left panel) treatment with Sorafinib with (Right panel) the response to a different inhibitor of the B-Raf pathway as per aspect of an embodiment of the present invention.

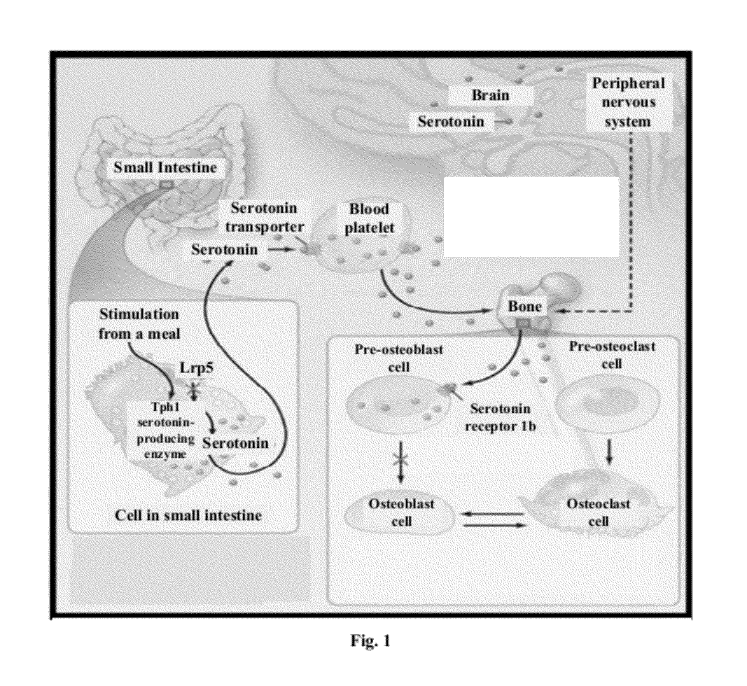

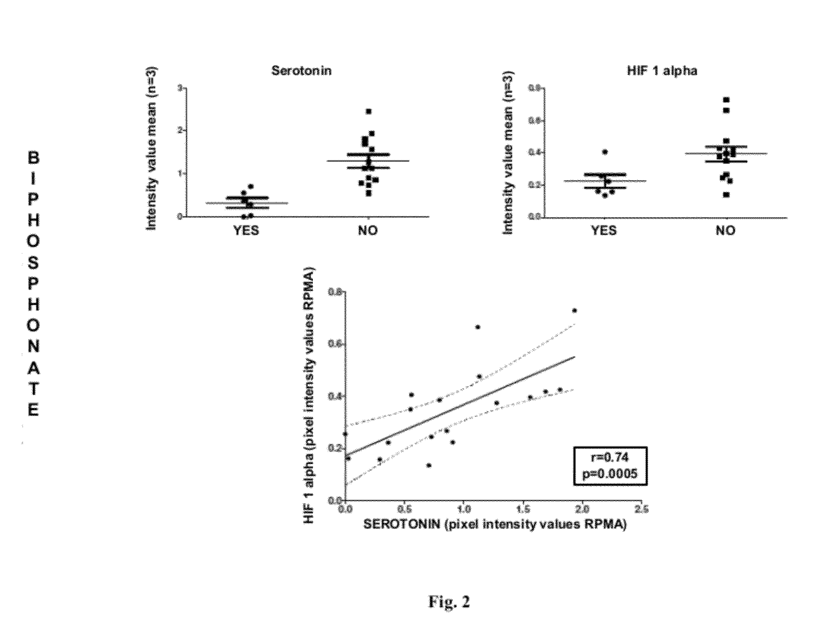

### DETAILED DESCRIPTION OF EMBODIMENTS

Aspects of embodiments include a method of treating a mammalian patient diagnosed with bone disease comprising delivering to the tissues of said patient or administering to said patient a therapeutically effective amount of one or more compounds, and wherein said one or more compounds comprises one or more of a tyrosine kinase inhibitor, a selective serotonin reuptake inhibitor (SSRI), a heterocyclic antidepressant, a monoamine oxidase inhibitor, an antidepressant, an anti-anxiety compound, an anti-epileptic, and an antibody.

Aspects of embodiments include a bone disease including one or more of brittle bone disease, multiple myeloma, osteogenesis imperfecta (OI), osteolytic bone disease, amyloidosis, monoclonal gammopathy, alterations in bone marrow hematopoietic precursor cells, myelodysplasia, or bone metastasis.

Aspects of embodiments include monoamine oxidase inhibitors including one or more of a selective monoamine oxidase inhibitor, a monoamine oxidase A inhibitor, a monoamine oxidase B inhibitor or a nonselective monoamine oxidase inhibitor.

In a further teaching, embodiments include conjoint administration of a therapeutically effective amount of one or more therapeutic agents or therapeutic treatments selected from the group consisting of an autophagy inhibitor, a non-chemotherapeutic agent, a chemotherapeutic agent, an angiogenesis inhibitor, a bone breakdown inhibitor, an osteoclast or osteoblast activity inhibitor, and an immune signal modulator.

Embodiments include tissues wherein, subsequent to treatment, serotonin is reduced in one or more of platelet cells, gastrointestinal cells, neural cells, immune cells, bone marrow microenvironment cells or cancer cells.

Embodiments include delivering said one or more compounds in an effective amount to modulate one or more of bone cell activity, stem cell activity, gastrointestinal cell activity, cancer cell activity, platelet cell activity, or neural cell activity.

Aspects of embodiments include a mammal that may be a human.

Embodiments include therapeutically effective amounts of one or more compounds that slow or arrest the growth of bone disease.

Embodiments include compounds that are independently administered orally, topically, subcutaneously, parenterally, transdermally, mucosally, rectally, intranasally, via inhalation, via insufflation, via a patch, via application to the site of the tumor or tumor bed, via installation into a wound, by buccal, or by sublingual administration.

Aspects of embodiments include a method of detecting the presence of bone disease in a mammal by machine-assaying detectable serotonin in a biological sample of the mammal, comparing the detectable serotonin in the biological sample with a positive or negative control; and identifying the presence of bone disease based on said detectable serotonin.

Aspects of embodiments include a method of detecting the presence of bone disease wherein said bone disease includes one or more of brittle bone disease, multiple myeloma, osteogenesis imperfecta (OI), osteolytic bone disease, amyloidosis, monoclonal gammopathy, alterations in bone marrow hematopoietic precursor cells, myelodysplasia, or bone metastasis.

Aspects of embodiments include a method of detecting the presence of bone disease wherein a biological sample includes one or more of isolated bone marrow aspirate, isolated bone tissue, isolated blood plasma, isolated blood serum, isolated blood cells, or isolated whole blood.

Additional aspects of embodiments include a method of detecting the presence of bone disease further comprising treating a mammal based on said identification of the presence of bone disease.

Aspects of embodiments include a method of detecting the presence of bone disease further comprising regulating bone remodeling in a subject including modulating serotonin in the subject based on detection.

An aspect of an embodiment includes a method of detecting the presence of bone disease in a mammal by assaying and/or performing one or more of post translational modification of signaling proteins, caspase cleavage, poly (ADP-ribose) polymerase (PARP) cleavage or dye exclusion/uptake, reverse phase microarray (RPMA), ELISA, flow cytometry, Immunohistochemistry, Immunoassay, high resolution mass spectroscopy, suspension bead array, or Western blot.

An aspect of an embodiment includes a method of treating bone disease in a subject by

administering an autophagy pathway inhibitor and administering one or more of a serotonin modulator, a tyrosine kinase inhibitor, an antidepressant, an anti-anxiety compound, an antiepileptic, a monoamine oxidase inhibitor, an antibody, a non-chemotherapeutic agent or a bisphosphonate wherein the bone disease includes one or more of monoclonal gammopathy of unknown significance (MGUS), premalignant bone marrow cells and multiple myeloma.

An aspect of an embodiment includes a method of treating bone disease in a subject including wherein an autophagy pathway inhibitor includes a 4-Amino-quinoline.

An aspect of an embodiment includes a method of treating bone disease in a subject including wherein an autophagy pathway inhibitor includes one or more of a 4-Amino-2-Alkyl-quinoline, a 2-Methyl-quinoline, a 4-Amino-3-Bromo-quinoline, a 4-Amino-3-Chloro-quinoline, a 4-Amino-6-Bromo-quinoline, a 4-Amino-7-Bromo-quinoline, or a 4-Amino-8-Bromo-quinoline.

An aspect of an embodiment includes a method of treating bone disease in a subject including wherein a bisphosphonate includes one or more of alendronate, pamidronate or zoledronic acid.

An aspect of an embodiment includes an autophagy pathway inhibitor that may be chloroquine.

Teachings include a method of treating bone disease including by selecting a patient or subject suffering from or at risk for bone disease.

Teachings include machines that are man-made articles of manufacture.

Embodiments relate to methods of treating disease in subjects. Other embodiments relate to articles of manufacture useful in treating disease, methods of making therapeutic compositions, combinations of therapeutic compositions, methods of administering therapeutic compositions, and dosages of therapeutic compositions.

In an aspect of an embodiment, a process to treat myelodysplasia includes methods of treating monoclonal gammopathy of unknown significance (MGUS), processes to treat multiple myeloma, processes to treat bone dysplasia and processes to treat abnormalities in bone.

In one aspect of an embodiment, a process, including a process to treat monoclonal gammopathy of unknown significance (MGUS), may include administering to a patient a therapeutically effective amount of a quinoline compound and a tyrosine kinase inhibitor.

In one aspect of an embodiment, a process, including a process to treat monoclonal gammopathy of unknown significance (MGUS), may include administering to a patient a therapeutically effective amount of a 4-aminoquinoline compound and a tyrosine kinase inhibitor.

In one embodiment, a process to modulate bone remodeling in a subject may include modulating serotonin in a subject. In an additional embodiment, said process to modulate bone remodeling may include increasing or decreasing bone density.

In another embodiment, a process to modulate bone remodeling in a subject may include administering to a subject a serotonin modulator in an effective amount wherein serotonin levels in a subject may be modulated. In a further teaching, a process to modulate bone remodeling produces plasma concentrations of platelet-derived serotonin. In another aspect of an embodiment, a process to modulate bone remodeling in a subject may include administering to a subject a medicament in an effective amount wherein serotonin levels in a subject are modulated.

In still another embodiment a process for modulating bone dysplasia, bone metastasis, treating monoclonal gammopathy of unknown significance (MGUS), premalignant bone marrow cells, or treating multiple myeloma may include administering a serotonin modulator to a subject in an effective amount. Said serotonin modulator may include a tyrosine kinase inhibitor, a selective serotonin reuptake inhibitor, a heterocyclic antidepressant, a monoamine oxidase inhibitor, an antidepressant, an anti-anxiolytic, an antiepileptic, or an antibody.

In an aspect of an embodiment, an article of manufacture may include at least one vessel containing purified chloroquine and purified HA14-1, instructions for the use of chloroquine and HA14-1 for the treatment monoclonal gammopathy of unknown significance (MGUS), multiple myeloma, bone dysplasia and/or abnormalities in bone, the treatment comprising (a) identifying a patient suspected of having said disease, and (b) administering an effective amount of chloroquine and HA 14-1 to the patient.

In an aspect of an embodiment, an article of manufacture may include a label that indicates the contents of the package may be used to treat at least one of multiple monoclonal gammopathy of unknown significance (MGUS), bone metastasis, multiple myeloma, bone dysplasia and/or abnormalities in bone, packaging material, and contained within the packing material purified chloroquine (or other 4-amino quinoline), and at least one of a purified tyrosine kinase inhibitor, a purified selective serotonin reuptake inhibitor (SSRI), a purified heterocyclic antidepressant, a purified monoamine oxidase inhibitor, a purified antidepressant, a purified anti-anxiety compound, a purified anti-epileptic, and a purified antibody.

In an aspect of an embodiment a method of treating bone disease may include administering a serotonin modulator to a subject. In an aspect of an embodiment, a method may include treating wherein said treating includes at least one of increasing bone density, decreasing bone density, maintaining bone density, or regulating elements associated with bone marrow.

In an aspect of an embodiment, a method of regulating may include altering at least one of preneoplastic differentiation of bone marrow cells, neoplastic angiogenesis of bone marrow cells, or bone marrow stem cell function.

In an aspect of an embodiment, a method of regulating may include wherein serotonin modulator alter at least one of the ratio of serotonin in platelets to plasma, concentration of serotonin in the bone marrow, serotonin receptor activity of cells within the bone marrow, production of serotonin by cells associated with the bone, intracellular signaling pathways associated with serotonin.

In an aspect of an embodiment, a method of treating bone disease may include altering at least one of administering a serotonin modulator to a subject. In a further embodiment the method of treating bone disease may include wherein said treating comprises at least one of the following increasing bone density, decreasing bone density, maintaining bone density; and regulating elements associated with bone marrow.

In an aspect of an embodiment, a method of regulating may include altering at least one of the following: preneoplastic differentiation of bone marrow cells, neoplastic angiogenesis of bone marrow cells, or bone marrow stem cell function.

In an aspect of an embodiment, serotonin modulator may alter at least one of the ratio of serotonin in platelets to plasma, concentration of serotonin in the bone marrow, serotonin receptor activity of cells within the bone marrow, production of serotonin by cells associated with the bone, intracellular signaling pathways associated with serotonin,

In an aspect of an embodiment a serotonin modulator may include a least one of a tyrosine kinase inhibitor, a selective serotonin reuptake inhibitor (SSRI), a heterocyclic antidepressant, a monoamine oxidase inhibitor, an antidepressant, an antianxiety compound, an antiepileptic compound, an antibody

In an aspect of an embodiment, a monoamine oxidase inhibitor may include a selective monoamine oxidase inhibitor, a monoamine oxidase A inhibitor, a monoamine oxidase B inhibitor or a nonselective monoamine oxidase inhibitor.

In an aspect of an embodiment, a method of treating bone disease may include administering at least one of an autophagy inhibitor, a non-chemotherapeutic agent, and angiogenesis inhibitor, a bone breakdown inhibitor, an osteoclast or osteoblast activity inhibitor, and an immune signal modulator.

An aspect of an embodiment, the method of treating bone disease may include reducing serotonin from platelet cells, gastrointestinal cells, neural cells, immune cells, bone marrow microenvironment cells or cancer cells.

In an aspect of an embodiment, a “bone breakdown inhibitor” may be administered in an effective amount to modulate at least one of bone cell activity, stem cell activity, gastrointestinal cell activity, cancer cell activity, platelet cell activity, and neural cell activity.

In a further teaching, a method of treating bone disease includes bone diseases such as brittle bone disease, multiple myeloma, osteogenesis imperfecta, osteolytic bone disease, amyloidosis, monoclonal gammopathy, alterations in bone marrow hematopoetic precursor cells, and myelodysplasia.

In still another aspect, a method of treating bone disease encompasses treating at least one of a chordate, mammal, primate, and human subject.

Another embodiment may include diagnosing and/or treating myeloma cells, including wherein said cells are inhibited, suppressed, or killed to a greater extent as compared to the non-myeloma cells.

Still another aspect of an embodiment includes a method of diagnosing a subject for a bone disease including at least the steps of assaying a biological sample of the subject, determining the amount of serotonin in said biological sample and determining a disease state based on said amount of serotonin.

Still another aspect of an embodiment includes diagnostic methods wherein said biological sample comprises at least one of the bone marrow aspirate, tissue, blood, serum, whole blood, cells, and blood.

Still another aspect of an embodiment includes diagnostic methods including at least the steps of determining the amount of serotonin in a known normal sample, determining the amount of serotonin in said biological sample, comparing said amount of serotonin in said known normal sample to the amount of serotonin in said biological sample. In another aspect of an embodiment, diagnostic methods include treating a subject based on serotonin level determination and or modulating serotonin in the subject based on said diagnosing.

In a still further teaching, diagnostic methods include assays evaluating post translational modification of signaling proteins, caspase cleavage, poly(ADP-ribose) polymerase (PARP) cleavage or dye exclusion/uptake.

According to another aspect of an embodiment, diagnostic methods may include evaluating methods employing at least one of reverse phase microarray (RPMA), ELISA, flow cytometry, Immunohistochemistry, Immunoassay, western blot, high resolution mass spectroscopy, and suspension bead array.

In a further teaching, a method of treating monoclonal gammopathy of unknown significance (MGUS), premalignant bone marrow cells or multiple myeloma in a subject may include treating with an autophagy pathway inhibitor, and at least one of a tyrosine kinase inhibitor, a serotonin modulator, an antidepressant, an anti-anxiety compound, an antiepileptic, a monoamine oxidase inhibitor, an antibody, a non-chemotherapeutic agent and a bisphosphonate.

In a further teaching, an autophagy pathway inhibitor such as a 4-amino quinoline may be used.

In a further aspect of an embodiment, treatment methods may retard the progression from a pre-disease state to multiple myeloma. In a further teaching, a modulator may include a least one of a tyrosine kinase inhibitor, a selective serotonin reuptake inhibitor (SSRI), a heterocyclic antidepressant; a monoamine oxidase inhibitor, an antidepressant, an anti-anxiety compound, an anti-epileptic, and an antibody.

In a further embodiment, a monoamine oxidase inhibitor may include at least one of a selective monoamine oxidase inhibitor, a monoamine oxidase A inhibitor, a monoamine oxidase B inhibitor and a nonselective monoamine oxidase inhibitor.

An additional aspect of an embodiment may include a method of treating bone disease including treating with at least one of an autophagy inhibitor, a non-chemotherapeutic agent, an angiogenesis inhibitor, a bone breakdown inhibitor, a osteoclast or osteoblast activity inhibitor, and an immune signal modulator.

In a further aspect of an embodiment, a method of treating bone disease may include reducing serotonin from at least one of platelet cells, gastrointestinal cells, neural cells, immune cells, bone marrow microenvironment cells and cancer cells.

In a further aspect of an embodiment, administering a bone breakdown inhibitor may include administering in an effective amount to modulate at least one of bone cell activity, stem cell activity, gastrointestinal cell activity, cancer cell activity, platelet cell activity, and neural cell activity. In a further aspect of an embodiment, a method of treating bone disease may include treating at least one of brittle bone disease, multiple myeloma, osteogenesis imperfecta (OI), osteolytic bone disease, amyloidosis, monoclonal gammopathy, alterations in bone marrow hematopoetic precursor cells, and myelodysplasia.

According to an aspect of an embodiment, the treatment methods may include treating at least one of a chordate, mammal, primate, and human.

According to embodiments, myeloma cells are inhibited, suppressed, or killed to a greater extent as compared to the non-myeloma cells.

According to embodiments, methods may include selecting a subject in need of treatment.

According to embodiments, a method of diagnosing a subject for a bone disease may include at least one of assaying a biological sample of the subject, determining the amount of serotonin in said biological sample, and determining a disease state based on said amount of serotonin.

According to embodiments, a biological sample to be used in a method may include at least one of bone marrow aspirate, tissue, blood serum, whole blood, cells, and blood.

According to embodiments, diagnostic methods include determining the amount of serotonin in a known normal sample, determining the amount of serotonin in a biological sample, and comparing said amount of serotonin in said known normal sample to the amount of serotonin in said biological sample. According to embodiments, diagnostic methods further include treating a subject based on said determination.

According to embodiments, regulating bone remodeling in the subject may include at least one of modulating serotonin in the subject based on said diagnosing.

According to embodiments, the assaying step may include at least one of assays evaluating post translational modification of signaling proteins, caspase cleavage, poly(ADP-ribose) polymerase (PARP) cleavage or dye exclusion/uptake.

According to embodiments, diagnostic methods may include at least one or more of evaluating where the evaluating includes at least one of reverse phase microarray (RPMA), ELISA, flow cytometry, Immunohistochemistry, Immunoassay, western blot, high resolution mass spectroscopy, and suspension bead array.

According to embodiments, a method of treating monoclonal gammopathy of unknown significance (MGUS), premalignant bone marrow cells or multiple myeloma in a subject may include at least one of administering an autophagy pathway inhibitor and at least one of a tyrosine kinase inhibitor, a serotonin modulator, an antidepressant, an anti-anxiety compound, an antiepileptic, a monoamine oxidase inhibitor, an antibody, a non-chemotherapeutic agent and a bisphosphonate.

In a further teaching, an autophagy pathway inhibitor may be a 4-amino quinoline.

In a further teaching, a bisphosphonate may include at least one of alendronate, pamidronate and zoledronic acid.

In an aspect of an embodiment, the monoamine oxidase inhibitor may include at least one of a selective monoamine oxidase inhibitor, a monoamine oxidase A inhibitor, a monoamine oxidase B inhibitor or a nonselective monoamine oxidase inhibitor.

In an aspect of an embodiment, the method may include treating with at least one of an autophagy inhibitor, and angiogenesis inhibitor, a bone breakdown inhibitor, an osteoclast or osteoblast activity inhibitor, and in immune signal modulator.

In an aspect of an embodiment, a kit may include a vessel or vessels containing purified 4-amino quinoline (for example chloroquine) and at least one of at least one of a purified tyrosine kinase inhibitor, a purified selective serotonin reuptake inhibitor (SSRI), a purified heterocyclic antidepressant, a purified monoamine oxidase inhibitor, purified an antidepressant, a purified anti-anxiety compound, a purified anti-epileptic, and a purified antibody.

In a further teaching, a method of treating may include a route of administration wherein said route of the administration may include at least one of intramuscular, transdermally, transmucossally, rectally, orally, via nasal insufflation, intravenous administration, and via cerebrospinal fluid or lumbar injection.

According to an additional embodiment, the form of the medicament may include at least one of a lotion, patch, injectable, tablet, or nasal spray.

In an additional embodiment, chloroquine analogs may include at least one of Chloroquine (CQ), 7-chloro-4-[[4-(diethylamino)-1-methylbutyl]amino] quinoline phosphate (1:2), Chloroquine Phosphate, USP, Qualiquin (Quinine), Plaquenil (hydroxychloroquine), Aralen, Aralen Phosphate, Lariam, or 4-aminoquinoline compounds.

According to embodiments, tyrosine kinase inhibitors may include at least one of Azitinib, Bosutinib, Cediranib, Crizotinib, Damnacanthal, Dasatinib, Erlotnib, Gefitinib, Imatinib, Lapatinib, Lestaurtinib, Neratinib, Nilotinib, Regorafenib, Ruxolitinb, Semaxanib, Sunitinib, Toceranib, Tofacitinib, Vandetanib, or Vatalnib.

According to embodiments, serotonin modulators may include at least one of selective serotonin reuptake inhibitors (SSRIs). SSRIs may include at least one of Citalopram, Dapoxetine, Escitalopram, Fluoxetine, Fluoxetine, Paroxetine, or Sertraline.

Additional embodiments include tricyclic antidepressants (TCAs). TCAs include Amitriptyline, Butripyline, Clomipramine, Desipramine, Dawsey Letha, Doxepin, Nortriptyline, Protriptyline, or Trimipramine.

In another embodiment, monoamine oxidase inhibitors (MAOIs) may include at least one of selective monoamine oxidase inhibitors, non-selective monoamine oxidase inhibitors, monoamine oxidase-A (MAO-A) inhibitors (Metralindole, Resveratrol, Berberine, Coptisine, Minaprine, Brofaromine, Toloxatone, Moclobemide, or Pirlindole), monoamine oxidase-B (MAO-B), inhibitors (Lazabemide, Pargyline, Rasagiline, or Selegiline), Hydrazines (Benmoxin, Hydralazine, Iproclozide, Iproniazid, Iprozid, Ipronid, Rivivol, Propilniazida, Isocarboxazid, Isoniazid, Mebanazine, Nialamide, Octamoxin, Phenelzine, Pheniprazine, Phenoxypropazine, Pivalylbenzhydrazine, Procarbazine, Natulan, or Indicarb, Safrazine), or Non-Hydrazines (Caroxazone, Echinopsidine, Furazolidone, Linezolid (Zyvox, Zyvoxam, Zyvoxid), or Tranylcypromine).

In another aspect, monoamine oxidase inhibitors may include at least one of Valproic Acid, Diazepam, licorice, Siberian ginseng, Yerba Mate, or Yohimbe.

In another aspect, serotonin agonists may include at least one of 5-HT_{1A} agonists (buspirone, gepirone, and tandospirone), 5-HT1B receptor agonists (sumatriptan, rizatriptan, and naratriptan), 5-HT_{1D} receptor agonists (sumatriptan, rizatriptan, and naratriptan), 5-HT_{1F} receptor agonist (Lasmiditan), 5-HT_{2A} receptor agonists (LSD, mescaline, psilocin, DMT, and 2C-B), 5-HT_{2C} receptor agonists (Lorcaserin), 5-HT₄ receptor agonist, and 5-HT₇ receptor agonists.

In another aspect, serotonin antagonists may include at least one of 5-HT1A antagonists, 5-HT1B receptor antagonists, 5-HT1D receptor antagonists, 5-HT1F receptor antagonists, 5-HT2A receptor antagonists, 5-HT2C receptor antagonists, 5-HT4 receptor antagonists, and 5-HT7 receptor antagonists.

According to embodiments, at least one process may include at least one of selecting or identifying a patient in need of treatment. According to embodiments a patient in need of treatment may be selected or identified as a patient presently diagnosed as having monoclonal gammopathy of unknown significance, premalignant bone, multiple myeloma, bone dysplasia, myeloma, amyloidosis, or myelodysplasia. According to embodiments, patients presently diagnosed may be diagnosed or identified via methods well known the skilled artisans. Such methods well known to skilled artisans may include physical examination, immunological detection methods, polymerase chain reaction (PCR)-based methods, reverse transcriptase-PCR (RT-PCR)-based methods, Southern, Northern, or Western analysis, flow cytometry, reverse phrase protein microarray (RPMA), proteomics, genomics, radiological testing processes or combinations thereof.

Embodiments may include at least one of methods of treating monoclonal gammopathy of unknown significance (MGUS). Additional embodiments include methods of treating multiple myeloma. Still other embodiments may include at least one of methods of treating myelodysplasia.

In another embodiment, potential therapeutics may include at least one of molecular inhibitors (e.g. Sunitinib, Dasatinib, Erlotinib), chemotherapeutics (e.g. Dexamethasone, Rapamycin, Bc1-2 inhibitor), or exogenous ligands (e.g. SCF, IGF-1 and/or cytokines (e.g. IL-6). Ideally, the potential therapeutics may target a wide range of growth, prosurvival, autophagy and angiogenesis-related pathways. Exemplary candidate therapeutics may include at least one of Avastin (bevacizumab), Gleevec (imatinib), Lapatinib, Iressa, Tarceva, Sutent (Sunitinib), Dasatinib (Sprycel), Nexavar (Sorafenib), Revlimid, Cucurbitacin I, A77 1726, AG 490, AG 1296, AGL 2043, Bcr-abl inhibitor, HNMPA-(AM)3, IGF-IR inhibitor, Lck inhibitor, LFM-A13, TGFβ inhibitor, CD20 antibody, Bortezomib, Carfilzomib, Chloroquine, Dasatinib, Dexamethasone, Erlotinib, Gefitinib, BCL-inhibitor, Honokiol, IGF-IR inhibitor II, Imatinib, Lapatinib, Mekl & 2 inhibitor, Melatonin, Midostaurin, Nilotinib, NVP-TKI258-CU-2, Nilotinib, Panobinostat, RAD, Rapamycin, Resveratrol, Sorafenib, Sunitinib, IL-6 ligand, IGF-1 ligand and SCF/C-kit ligand.

In another embodiment, a method of treating bone disease in a subject may include at least one of administering a serotonin modulator to a subject alone or in combination with other therapies.

In another embodiment, regulating elements residing in the bone marrow may include altering the proliferation, genetic stability, survival, and/or function of cellular elements residing in bone or bone marrow.

In another embodiment, treating may include regulating bone, for example bone cells, bone marrow cells, bone stroma, gut cells, platelet cells, brain cells, and ventromedial cells.

In another embodiment, modulating of serotonin may include at least one of administering a serotonin modulator in an effective amount to modulate serotonin levels or the effects mediated by serotonin.

In another embodiment, serotonin modulators may include at least one of bone breakdown inhibitors, and inhibitors that modulate osteoclast and osteoblast activity.

In another embodiment, serotonin modulators may include at least one of a tyrosine kinase inhibitor, a selective serotonin reuptake inhibitor (SSRI), a heterocyclic antidepressant, a monoamine oxidase inhibitor, an antidepressant, an anti-anxiety compound, an anti-epileptic and an antibody.

In another embodiment, treating may include treating with at least one of an agent that modulates the signaling pathways associated with the action of serotonin, an agent that may be synergistic or an agent that may be additive with serotonin modulation.

In another embodiment, cells may be regulated by at least one of preneoplastic differentiation, neoplastic angiogenesis or stem cell function. For example, bone marrow stem cell function includes differentiation of pre-osteoblasts into osteoblasts.

In another embodiment, regulating elements residing in the bone marrow may include at least one of altering the proliferation, genetic stability, survival, and/or function of cellular elements residing in bone or bone marrow.

In another embodiment, preneoplastic and neoplastic disease of the bone may be treated by at least one of modulation of the bone neuro-endocrine axis.

In another embodiment, treatment of bone disease may include treatment that blocks at least one member of the autophagy pathway in order to modulate autophagy regulation.

In an embodiment, a 4-amino-quinoline may include at least one 2-alkyl-quinoline. In a further embodiment, at least one 2-alkyl-quinoline may include at least one of 4-amino-6,8-difluoro-2-methylquinoline, 4-amino-5,8-difluoro-2-methylquinoline, 4-amino-5,7-difluoro-2-methylquinoline, 4-Amino-2-propylquinoline, 4-Amino-7-methoxy-2-propylquinoline, 4-Amino-8-methoxy-2-propylquinoline, 4-Amino-6-fluoro-2-propylquinoline, 4-Amino-7-fluoro-2-propylquinoline, 4-Amino-8-fluoro-2-propylquinoline, 4-Amino-6-chloro-2-propylquinoline, 4-Amino-7-chloro-2-propylquinoline, 4-Amino-8-chloro-2-propylquinoline, 4-Amino-6-bromo-2-propylquinoline, 4-Amino-7-bromo-2-propylquinoline, 4-Amino-8-bromo-2-propylquinoline, 4-Amino-2-propyl-6-trifluoromethylquinoline, 4-Amino-2-propyl-7-trifluoromethylquinoline, 4-Amino-2-propyl-8-trifluoromethylquinoline, 4-Amino-5,7-dichloro-2-propylquinoline, 4-Amino-5,8-dichloro-2-propylquinoline, 4-Amino-6,7-dichloro-2-propylquinoline, 4-Amino-6,8-dichloro-2-propylquinoline, 4-Amino-7,8-dichloro-2-propylquinoline, 4-Amino-5-chloro-8-methyl-2-propylquinoline, 4-Amino-6-chloro-8-methyl-2-propylquinoline, 4-Amino-7-chloro-8-methyl-2-propylquinoline, 4-Amino-8-chloro-6-methyl-2-propylquinoline, 4-Amino-2-methyl-6-trifluoromethoxyquinoline, 4-Amino-2-methyl-7-trifluoromethoxyquinoline, 4-Amino-2-methyl-8-trifluoromethoxyquinoline, 4-Amino-7,8-difluoro-2-methylquinoline, 4-Amino-6-bromo-2,8-dimethylquinoline, 4-Amino-7-bromo-2,8-dimethylquinoline, 4-Amino-8-bromo-2,6-dimethylquinoline, 4-Amino-5-chloro-8-methoxy-2-methylquinoline, 4-Amino-6-chloro-8-methoxy-2-methylquinoline, 4-Amino-8-chloro-5-methoxy-2-methylquinoline, 4-Amino-8-bromo-2-methylquinoline, 4-Amino-6-bromo-2-methylquinoline, 4-Amino-2-propyl-6-trifluoromethoxyquinoline, 4-Amino-2-propyl-7-trifluoromethoxyquinoline, 4-Amino-6,8-dibromo-2-methylquinoline, 4-Amino-2-propyl-8-trifluoromethoxyquinoline, 4-Amino-2,6-dimethylquinoline, 4-Amino-6,8-difluoro-2-propylquinoline, 4-Amino-2,8-dimethylquinoline, 4-Amino-7,8-difluoro-2-propylquinoline, 4-Amino-6-bromo-8-methyl-2-propylquinoline, 4-Amino-7-bromo-8-methyl-2-propylquinoline, 4-Amino-7-bromo-6-methyl-2-propylquinoline, 4-Amino-6-fluoro-2-methylquinoline, 4-Amino-8-bromo-6-methyl-2-propylquinoline, 4-Amino-6-chloro-8-methoxy-2-propylquinoline, 4-Amino-8-chloro-5-methoxy-2-propylquinoline, 4-Amino-6-methoxy-2-methylquinoline, 4-Amino-2-methyl-8-(trifluoromethyl)quinolone, 4-Amino-2-methyl-7-(trifluoromethyl)quinolone, 4-Amino-6,8-dichloro-2-methylquinoline, 4-Amino-7,8-dichloro-2-methylquinoline, 4-Amino-5,7-dichloro-2-methylquinoline, 4-Amino-5,8-dichloro-2-methylquinoline, 4-Amino-2,5,7-trimethylquinoline, 4-Amino-2,5,8-trimethylquinoline, 4-Amino-2,6,8-trimethylquinoline, 4-Amino-2,7,8-trimethylquinoline, 4-Amino-2-methyl-6-trifluoromethylquinoline, 4-Amino-5-chloro-2,8-dimethylquinoline, 4-Amino-6,7-dichloro-2-methylquinoline, 4-Amino-6-chloro-2,8-dimethylquinoline, 4-Amino-6-chloro-2-methylquinoline, 4-Amino-6-ethoxy-2-methylquinoline, 4-Amino-6-ethyl-2-methylquinoline, 4-Amino-7-bromo-2-methylquinoline, 4-Amino-7-chloro-2,8-dimethylquinoline, 4-Amino-7-chloro-2-methylquinoline, 4-Amino-7-fluoro-2-methylquinoline, 4-Amino-7-methoxy-2-methylquinoline, 4-Amino-8-chloro-2,6-dimethylquinoline, 4-Amino-8-chloro-2-methylquinoline, 4-Amino-8-ethyl-2-methylquinoline, 4-Amino-8-fluoro-2-methylquinoline, and 4-Amino-8-methoxy-2-methylquinoline.

In an embodiment, a 4-amino-quinoline may include at least one 2-Methyl-quinoline. In a further embodiment, at least one 2-Methyl-quinoline may include at least one of 4-amino-6,8-difluoro-2-methylquinoline, 4-amino-5,8-difluoro-2-methylquinoline, 4-amino-5,7-difluoro-2-methylquinoline, 4-Amino-2-methyl-6-trifluoromethoxyquinoline, 4-Amino-2-methyl-7-trifluoromethoxyquinoline, 4-Amino-2-methyl-8-trifluoromethoxyquinoline, 4-Amino-7,8-difluoro-2-methylquinoline, 4-Amino-6-bromo-2,8-dimethylquinoline, 4-Amino-7-bromo-2,8-dimethylquinoline, 4-Amino-8-bromo-2,6-dimethylquinoline, 4-Amino-5-chloro-8-methoxy-2-methylquinoline, 4-Amino-6-chloro-8-methoxy-2-methylquinoline, 4-Amino-8-chloro-5-methoxy-2-methylquinoline, 4-Amino-8-bromo-2-methylquinoline, 4-Amino-6-bromo-2-methylquinoline, 4-Amino-6,8-dibromo-2-methylquinoline, 4-Amino-2,6-dimethylquinoline, 4-Amino-2,8-dimethylquinoline, 4-Amino-6-fluoro-2-methylquinoline, 4-Amino-6-methoxy-2-methylquinoline, 4-Amino-2-methyl-8-(trifluoromethyl)quinolone, 4-Amino-2-methyl-7-(trifluoromethyl)quinolone, 4-Amino-6,8-dichloro-2-methylquinoline, 4-Amino-7,8-dichloro-2-methylquinoline, 4-Amino-5,7-dichloro-2-methylquinoline, 4-Amino-5,8-dichloro-2-methylquinoline, 4-Amino-2,5,7-trimethylquinoline, 4-Amino-2,5,8-trimethylquinoline, 4-Amino-2,6,8-trimethylquinoline, 4-Amino-2,7,8-trimethylquinoline, 4-Amino-2-methyl-6-trifluoromethylquinoline, 4-Amino-5-chloro-2,8-dimethylquinoline, 4-Amino-6,7-dichloro-2-methylquinoline, 4-Amino-6-chloro-2,8-dimethylquinoline, 4-Amino-6-chloro-2-methylquinoline, 4-Amino-6-ethoxy-2-methylquinoline, 4-Amino-6-ethyl-2-methylquinoline, 4-Amino-7-bromo-2-methylquinoline, 4-Amino-7-chloro-2,8-dimethylquinoline, 4-Amino-7-chloro-2-methylquinoline, 4-Amino-7-fluoro-2-methylquinoline, 4-Amino-7-methoxy-2-methylquinoline, 4-Amino-8-chloro-2,6-dimethylquinoline, 4-Amino-8-chloro-2-methylquinoline, 4-Amino-8-ethyl-2-methylquinoline, 4-Amino-8-fluoro-2-methylquinoline, and 4-Amino-8-methoxy-2-methylquinoline.

In an embodiment, a 4-amino-quinoline may include at least one 3-Bromo-quinoline. In a further embodiment, at least one 3-Bromo-quinoline may include at least one of 4-Amino-3-bromoquinoline, 4-Amino-3-bromo-6-methylquinoline, 4-Amino-3-bromo-7-methylquinoline, 4-Amino-3-bromo-8-methylquinoline, 4-Amino-3-bromo-6-ethylquinoline, 4-Amino-3-bromo-8-ethylquinoline, 4-Amino-3-bromo-6-methoxyquinoline, 4-Amino-3-bromo-7-methoxyquinoline, 4-Amino-3-bromo-8-methoxyquinoline, 4-Amino-3-bromo-6-ethoxyquinoline, 4-Amino-3-bromo-6-fluoroquinoline, 4-Amino-3-bromo-7-fluoroquinoline, 4-Amino-3-bromo-8-fluoroquinoline, 4-Amino-3-bromo-6-chloroquinoline, 4-Amino-3-bromo-8-chloroquinoline, 4-Amino-3,6-dibromoquinoline, 4-Amino-3,7-dibromoquinoline, 4-Amino-3,8-dibromoquinoline, 4-Amino-3-bromo-6-trifluoromethylquinoline, 4-Amino-3-bromo-7-trifluoromethylquinoline, 4-Amino-3-bromo-8-trifluoromethylquinoline, 4-Amino-3-bromo-6-trifluoromethoxyquinoline, 4-Amino-3-bromo-7-trifluoromethoxyquinoline, 4-Amino-3-bromo-8-trifluoromethoxyquinoline, 4-Amino-3-bromo-5,7-dimethylquinoline, 4-Amino-3-bromo-5,8-dimethylquinoline, 4-Amino-3-bromo-6,7-dimethylquinoline, 4-Amino-3-bromo-6,8-dimethylquinoline, 4-Amino-3-bromo-7,8-dimethylquinoline, 4-Amino-3-bromo-5,7-difluoroquinoline, 4-Amino-3-bromo-5,8-difluoroquinoline, 4-Amino-3-bromo-6,8-difluoroquinoline, 4-Amino-3-bromo-7,8-difluoroquinoline, 4-Amino-3-bromo-5,7-dichloroquinoline, 4-Amino-3-bromo-5,8-dichloroquinoline, 4-Amino-3-bromo-6,7-dichloroquinoline, 4-Amino-3-bromo-6,8-dichloroquinoline, 4-Amino-3-bromo-7,8-dichloroquinoline, 4-Amino-3-bromo-5-chloro-8-methylquinoline, 4-Amino-3-bromo-6-chloro-8-methylquinoline, 4-Amino-3-bromo-7-chloro-8-methylquinoline, 4-Amino-3-bromo-8-chloro-6-methylquinoline, 4-Amino-3,6-dibromo-8-methylquinoline, 4-Amino-3,7-dibromo-6-methylquinoline, 4-Amino-3,7-dibromo-8-methylquinoline, 4-Amino-3,8-dibromo-6-methylquinoline, 4-Amino-3-bromo-5-chloro-8-methoxyquinoline, 4-Amino-3-bromo-6-chloro-8-methoxyquinoline, and 4-Amino-3-bromo-8-chloro-5-methoxyquinoline.

In an embodiment, a 4-amino-quinoline may include at least one 3-Chloro-quinoline. In a further embodiment, at least one 3-Chloro-quinoline may include at least one 3-Bromo-quinoline 4-Amino-3-chloroquinoline, 4-Amino-3-chloro-6-methylquinoline, 4-Amino-3-chloro-7-methylquinoline, 4-Amino-3-chloro-8-methylquinoline, 4-Amino-3-chloro-6-ethylquinoline, 4-Amino-3-chloro-8-ethylquinoline, 4-Amino-3-chloro-6-methoxyquinoline, 4-Amino-3-chloro-7-methoxyquinoline, 4-Amino-3-chloro-8-methoxyquinoline, 4-Amino-3-chloro-6-ethoxyquinoline, 4-Amino-3-chloro-6-fluoroquinoline, 4-Amino-3-chloro-7-fluoroquinoline, 4-Amino-3-chloro-8-fluoroquinoline, 4-Amino-3,6-dichloroquinoline, 4-Amino-3,8-dichloroquinoline, 4-Amino-6-bromo-3-chloroquinoline, 4-Amino-7-bromo-3-chloroquinoline, 4-Amino-8-bromo-3-chloroquinoline, 4-Amino-3-chloro-6-trifluoromethylquinoline, 4-Amino-3-chloro-7-trifluoromethylquinoline, 4-Amino-3-chloro-8-trifluoromethylquinoline, 4-Amino-3-chloro-6-trifluoromethoxyquinoline, 4-Amino-3-chloro-7-trifluoromethoxyquinoline, 4-Amino-3-chloro-8-trifluoromethoxyquinoline, 4-Amino-3-chloro-5,7-dimethylquinoline, 4-Amino-3-chloro-5,8-dimethylquinoline, 4-Amino-3-chloro-6,7-dimethylquinoline, 4-Amino-3-chloro-6,8-dimethylquinoline, 4-Amino-3-chloro-7,8-dimethylquinoline, 4-Amino-3-chloro-5,7-difluoroquinoline, 4-Amino-3-chloro-5,8-difluoroquinoline, 4-Amino-3-chloro-6,8-difluoroquinoline, 4-Amino-3-chloro-7,8-difluoroquinoline, 4-Amino-3,5,7-trichloroquinoline, 4-Amino-3,5,8-trichloroquinoline, 4-Amino-3,6,7-trichloroquinoline, 4-Amino-3,6,8-trichloroquinoline, 4-Amino-3,7,8-trichloroquinoline, 4-Amino-3,5-dichloro-8-methylquinoline, 4-Amino-3,6-dichloro-8-methylquinoline, 4-Amino-3,7-dichloro-8-methylquinoline, 4-Amino-3,8-dichloro-6-methylquinoline, 4-Amino-6-bromo-3-chloro-8-methylquinoline, 4-Amino-7-bromo-3-chloro-6-methylquinoline, 4-Amino-7-bromo-3-chloro-8-methylquinoline, 4-Amino-8-bromo-3-chloro-6-methylquinoline, 4-Amino-3,5-dichloro-8-methoxyquinoline, 4-Amino-3,6-dichloro-8-methoxyquinoline, and 4-Amino-3,8-dichloro-5-methoxyquinoline.

In an embodiment, a 4-amino-quinoline may include 6-Bromo-quinolines. In a further embodiment, 6-Bromo-quinolines may include at least one of 4-Amino-6-bromo-2-phenylquinoline, 4-Amino-6-bromo-2-propylquinoline, 4-Amino-6-bromo-8-methylquinoline, 4-Amino-6-bromo-7-methylquinoline, 4-Amino-6-bromo-2,8-dimethylquinoline, 4-Amino-6-bromo-8-methyl-2-phenylquinoline, 4-Amino-6-bromo-2-methylquinoline, 4-Amino-6,8-dibromo-2-methylquinoline, 4-Amino-6-bromo-8-methyl-2-propylquinoline, 4-Amino-3,6-dibromoquinoline, 4-Amino-6-bromo-3-chloroquinoline, 4-Amino-3,6-dibromo-8-methylquinoline, 4-Amino-6-bromo-3-chloro-8-methylquinoline, 4-Amino-6-bromoquinoline-3-carboxylic acid ethyl ester, 4-Amino-6-bromo-8-methylquinoline-3-carboxylic acid ethyl ester, 4-Amino-6-bromoquinoline-3-carboxylic acid, 4-Amino-6-bromo-8-methylquinoline-3-carboxylic acid, and 4-Amino-6-bromoquinoline.

In an embodiment, a 4-amino-quinoline may include at least one 7-Bromo-quinoline. In a further embodiment, at least one 7-Bromo-quinoline may include at least one of 4-Amino-7-bromo-2-phenylquinoline, 4-Amino-7-bromo-2-propylquinoline, 4-Amino-7-bromo-8-methylquinoline, 4-Amino-7-bromo-6-methylquinoline, 4-Amino-7-bromo-2,8-dimethylquinoline, 4-Amino-7-bromo-8-methyl-2-phenylquinoline, 4-Amino-7-bromo-6-methyl-2-phenylquinoline, 4-Amino-7-bromo-8-methyl-2-propylquinoline, 4-Amino-7-bromo-6-methyl-2-propylquinoline, 4-Amino-3,7-dibromoquinoline, 4-Amino-7-bromo-3-chloroquinoline, 4-Amino-3,7-dibromo-6-methylquinoline, 4-Amino-7-bromo-3-chloro-6-methylquinoline, 4-Amino-3,7-dibromo-8-methylquinoline, 4-Amino-7-bromo-3-chloro-8-methylquinoline, 4-Amino-7-bromo-8-methylquinoline-3-carboxylic acid ethyl ester, 4-Amino-7-bromo-8-methylquinoline-3-carboxylic acid, 4-Amino-7-bromo-2-methylquinoline, and 4-Amino-7-bromoquinoline.

In an embodiment, a 4-amino-quinoline may include at least one 8-Bromo-quinoline. In a further embodiment, at least one 8-Bromo-quinoline may include at least one of 4-Amino-8-bromo-2-phenylquinoline, 4-Amino-8-bromo-2-propylquinoline, 4-Amino-8-bromo-6-methylquinoline, 4-Amino-8-bromo-2,6-dimethylquinoline, 4-Amino-8-bromo-6-methyl-2-phenylquinoline, 4-Amino-8-bromo-2-methylquinoline, 4-Amino-6,8-dibromo-2-methylquinoline, 4-Amino-8-bromo-6-methyl-2-propylquinoline, 4-Amino-3,8-dibromoquinoline, 4-Amino-8-bromo-3-chloroquinoline, 4-Amino-3,8-dibromo-6-methylquinoline, 4-Amino-8-bromo-3-chloro-6-methylquinoline, 4-Amino-8-bromoquinoline-3-carboxylic acid ethyl ester, 4-Amino-8-bromo-6-methylquinoline-3-carboxylic acid ethyl ester, 4-Amino-8-bromoquinoline-3-carboxylic acid, 4-Amino-8-bromo-6-methylquinoline-3-carboxylic acid, and 4-Amino-8-bromoquinoline.

In an aspect of an embodiment, a 4-amino-quinoline may include at least one Amino acid. In a further embodiment, at least one Amino acid may include at least one of 4-Aminoquinoline-6-carboxylic acid, 4-Aminoquinoline-7-carboxylic acid, 4-Aminoquinoline-3-carboxylic acid, 4-Amino-6-methylquinoline-3-carboxylic acid, 4-Amino-8-methylquinoline-3-carboxylic acid, 4-Amino-6-ethylquinoline-3-carboxylic acid, 4-Amino-8-ethylquinoline-3-carboxylic acid, 4-Amino-6-methoxyquinoline-3-carboxylic acid, 4-Amino-8-methoxyquinoline-3-carboxylic acid, 4-Amino-6-ethoxyquinoline-3-carboxylic acid, 4-Amino-6-fluoroquinoline-3-carboxylic acid, 4-Amino-7-fluoroquinoline-3-carboxylic acid, 4-Amino-8-fluoroquinoline-3-carboxylic acid, 4-Amino-6-chloroquinoline-3-carboxylic acid, 4-Amino-8-chloroquinoline-3-carboxylic acid, 4-Amino-6-bromoquinoline-3-carboxylic acid, 4-Amino-8-bromoquinoline-3-carboxylic acid, 4-Amino-6-(trifluoromethyl)quinoline-3-carboxylic acid, 4-Amino-7-(trifluoromethyl)quinoline-3-carboxylic acid, 4-Amino-5,7-dimethylquinoline-3-carboxylic acid, 4-Amino-5,8-dimethylquinoline-3-carboxylic acid, 4-Amino-6,8-dimethylquinoline-3-carboxylic acid, 4-Amino-7,8-dimethylquinoline-3-carboxylic acid, 4-Amino-5,7-difluoroquinoline-3-carboxylic acid, 4-Amino-5,8-difluoroquinoline-3-carboxylic acid, 4-Amino-6,8-difluoroquinoline-3-carboxylic acid, 4-Amino-7,8-difluoroquinoline-3-carboxylic acid, 4-Amino-5,7-dichloroquinoline-3-carboxylic acid, 4-Amino-5,8-dichloroquinoline-3-carboxylic acid, 4-Amino-6,8-dichloroquinoline-3-carboxylic acid, 4-Amino-7,8-dichloroquinoline-3-carboxylic acid, 4-Amino-5-chloro-8-methylquinoline-3-carboxylic acid, 4-Amino-6-chloro-8-methylquinoline-3-carboxylic acid, 4-Amino-7-chloro-8-methylquinoline-3-carboxylic acid, 4-Amino-8-chloro-6-methylquinoline-3-carboxylic acid, 4-Amino-6-bromo-8-methylquinoline-3-carboxylic acid, 4-Amino-7-bromo-8-methylquinoline-3-carboxylic acid, 4-Amino-8-bromo-6-methylquinoline-3-carboxylic acid, 4-Amino-5-chloro-8-methoxyquinoline-3-carboxylic acid, and 4-Amino-8-chloro-5-methoxyquinoline-3-carboxylic acid.

In an embodiment, a 4-amino-quinoline may include at least one Amino Ester. In a further embodiment, at least one Amino Esters may include at least one of 4-Aminoquinoline-3-carboxylic acid ethyl ester, 4-Amino-6-methylquinoline-3-carboxylic acid ethyl ester, 4-Amino-8-methylquinoline-3-carboxylic acid ethyl ester, 4-Amino-6-ethylquinoline-3-carboxylic acid ethyl ester, 4-Amino-8-ethylquinoline-3-carboxylic acid ethyl ester, 4-Amino-6-methoxyquinoline-3-carboxylic acid ethyl ester, 4-Amino-8-methoxyquinoline-3-carboxylic acid ethyl ester, 4-Amino-6-ethoxyquinoline-3-carboxylic acid ethyl ester, 4-Amino-6-fluoroquinoline-3-carboxylic acid ethyl ester, 4-Amino-7-fluoroquinoline-3-carboxylic acid ethyl ester, 4-Amino-8-fluoroquinoline-3-carboxylic acid ethyl ester, 4-Amino-6-chloroquinoline-3-carboxylic acid ethyl ester, 4-Amino-8-chloroquinoline-3-carboxylic acid ethyl ester, 4-Amino-6-bromoquinoline-3-carboxylic acid ethyl ester, 4-Amino-8-bromoquinoline-3-carboxylic acid ethyl ester, 4-Amino-6-(trifluoromethyl)quinoline-3-carboxylic acid ethyl ester, 4-Amino-7-(trifluoromethyl)quinoline-3-carboxylic acid ethyl ester, 4-Amino-8-(trifluoromethyl)quinoline-3-carboxylic acid ethyl ester, 4-Amino-5,7-dimethylquinoline-3-carboxylic acid ethyl ester, 4-Amino-5,8-dimethylquinoline-3-carboxylic acid ethyl ester, 4-Amino-6,8-dimethylquinoline-3-carboxylic acid ethyl ester, 4-Amino-7,8-dimethylquinoline-3-carboxylic acid ethyl ester, 4-Amino-5,7-difluoroquinoline-3-carboxylic acid ethyl ester, 4-Amino-5,8-difluoroquinoline-3-carboxylic acid ethyl ester, 4-Amino-6,8-difluoroquinoline-3-carboxylic acid ethyl ester, 4-Amino-7,8-difluoroquinoline-3-carboxylic acid ethyl ester, 4-Amino-5,7-dichloroquinoline-3-carboxylic acid ethyl ester, 4-Amino-5,8-dichloroquinoline-3-carboxylic acid ethyl ester, 4-Amino-6,8-dichloroquinoline-3-carboxylic acid ethyl ester, 4-Amino-7,8-dichloroquinoline-3-carboxylic acid ethyl ester, 4-Amino-5-chloro-8-methylquinoline-3-carboxylic acid ethyl ester, 4-Amino-6-chloro-8-methylquinoline-3-carboxylic acid ethyl ester, 4-Amino-7-chloro-8-methylquinoline-3-carboxylic acid ethyl ester, 4-Amino-8-chloro-6-methylquinoline-3-carboxylic acid ethyl ester, 4-Amino-6-bromo-8-methylquinoline-3-carboxylic acid ethyl ester, 4-Amino-7-bromo-8-methylquinoline-3-carboxylic acid ethyl ester, 4-Amino-8-bromo-6-methylquinoline-3-carboxylic acid ethyl ester, 4-Amino-5-chloro-8-methoxyquinoline-3-carboxylic acid ethyl ester, and 4-Amino-8-chloro-5-methoxyquinoline-3-carboxylic acid ethyl ester.

We have developed methods of ex vivo treatment of bone marrow aspirate samples (PCT/US2009/004608). We describe methods to screen a large series of kinase and cell signaling inhibitors in fresh, living patient's myeloma cells within the tumor microenvironment within 4 hours of collection. The technology provides a method to magnetically sort, in a multiplexed high throughput manner, cellular samples with concomitant analysis of plasma cells and non-plasma cells. Employing these methods we have compiled drug inhibitory data for 35 human MM bone marrow aspirate samples using ex vivo functional screening. This information has provided insights into new therapies or combinations of therapies for treatment of MM.

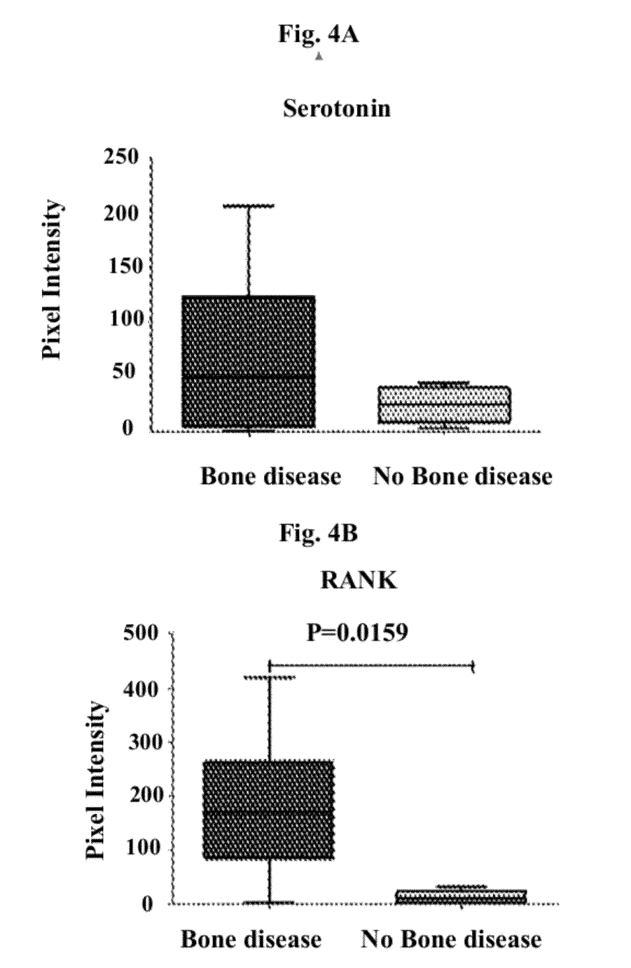

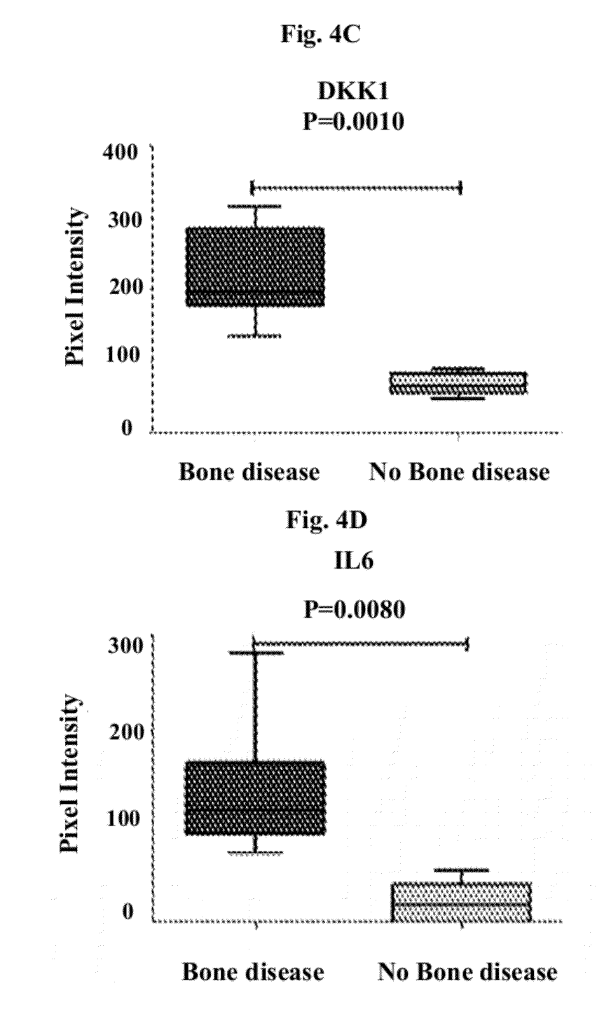

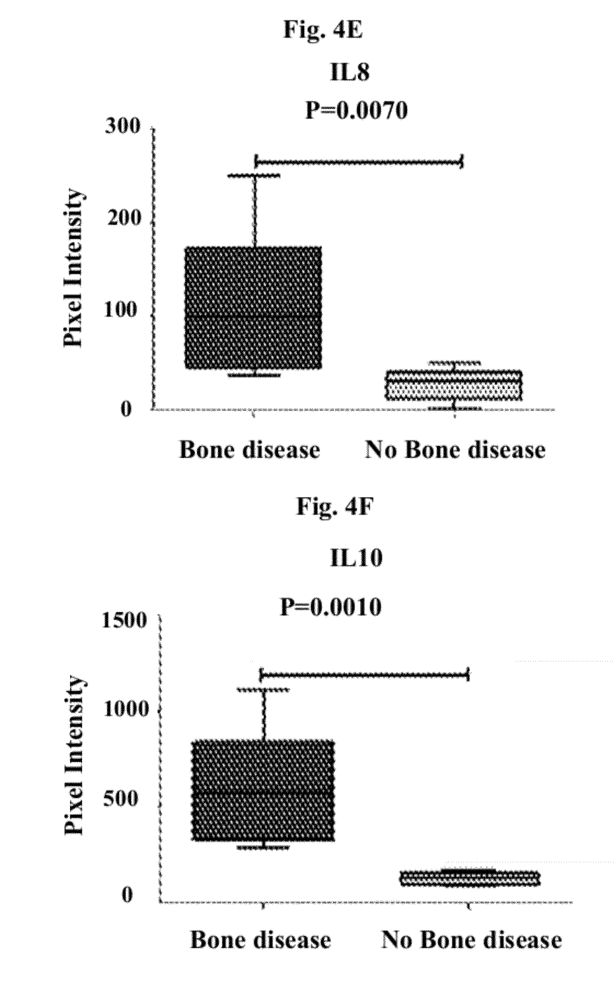

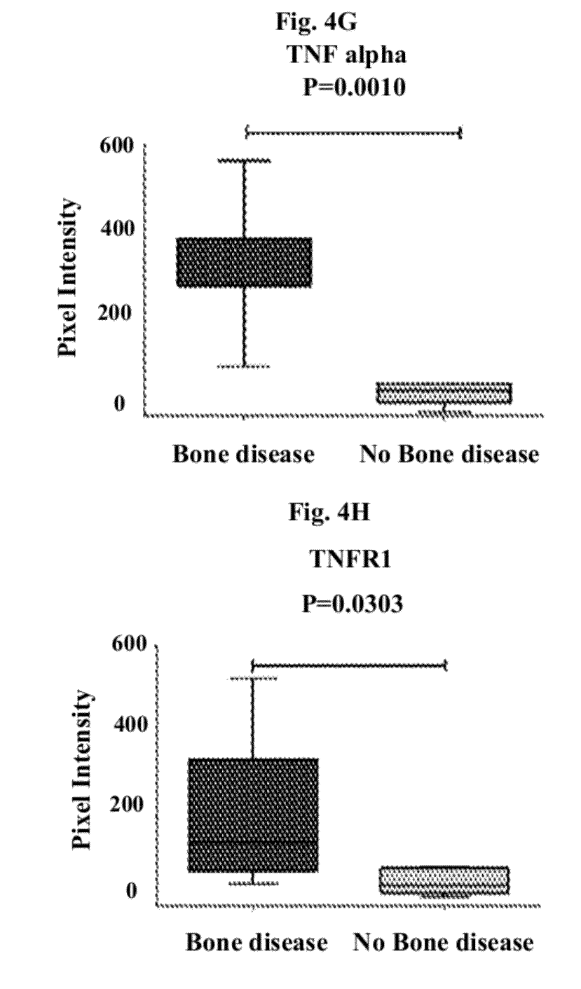

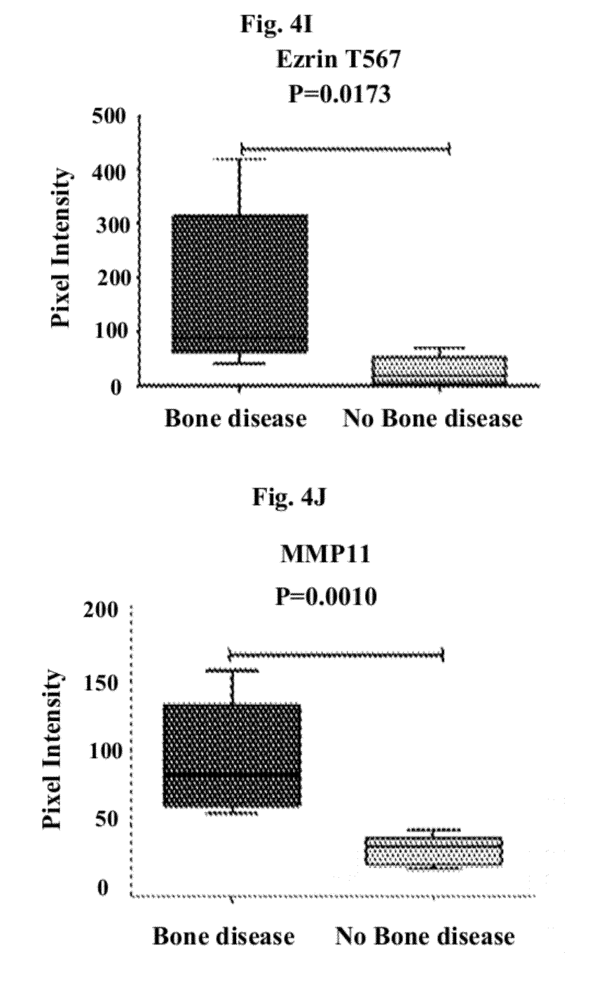

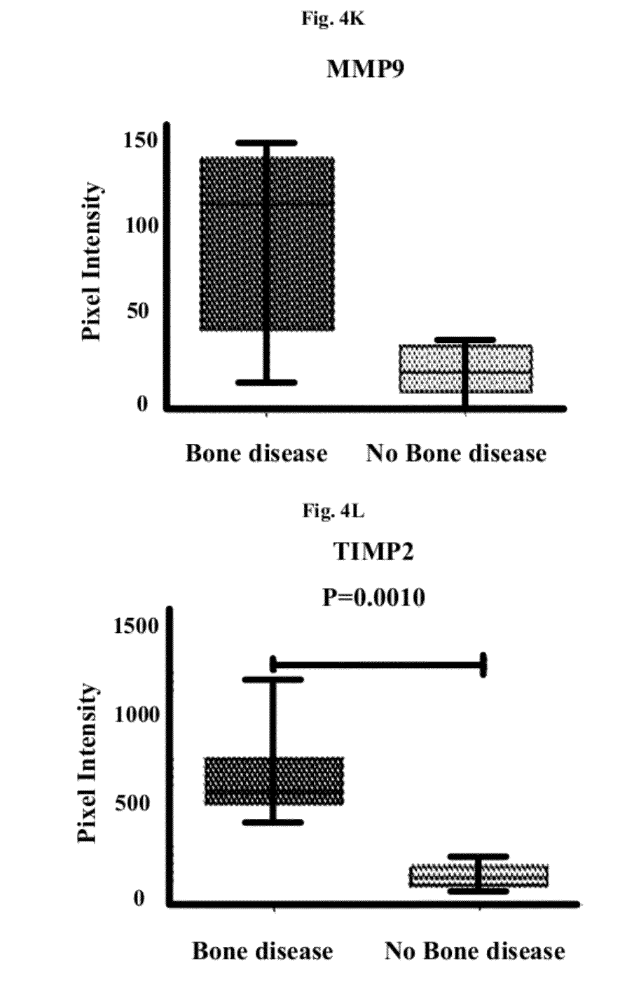

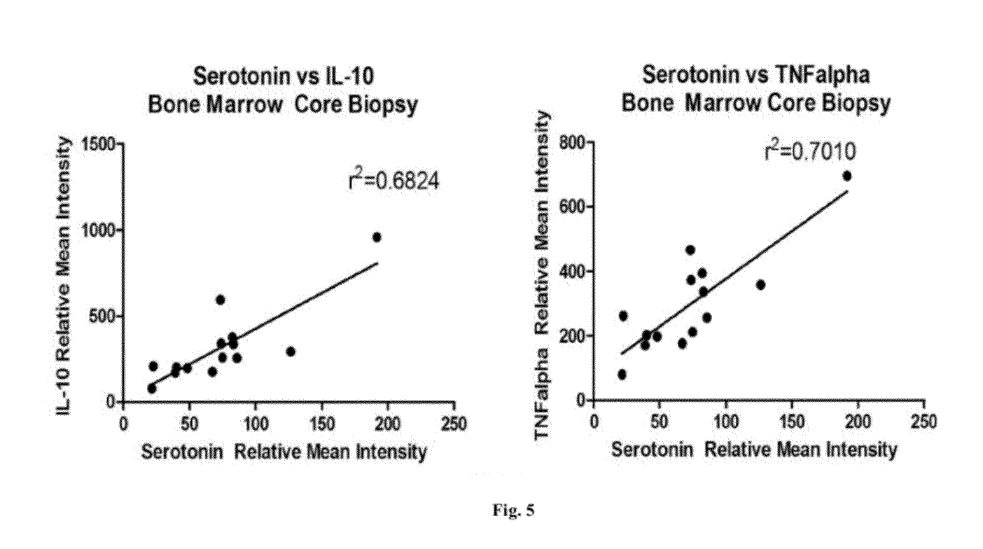

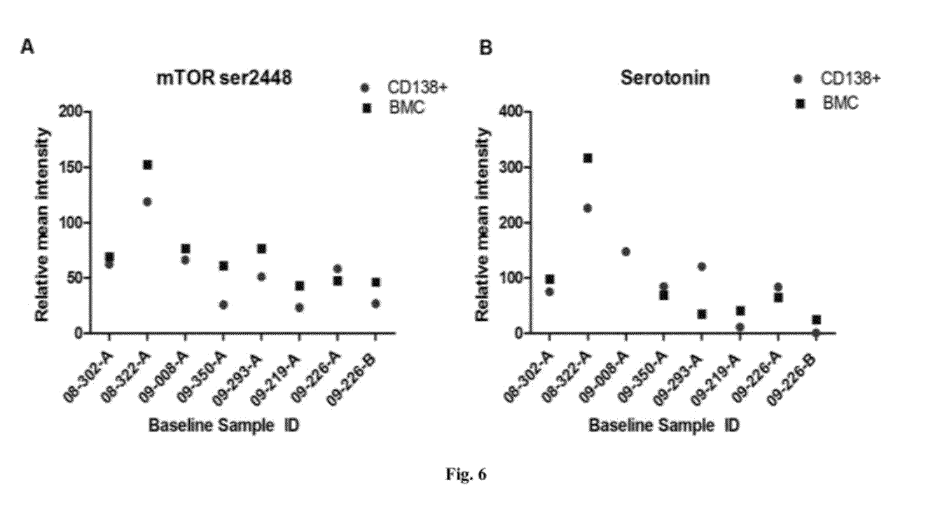

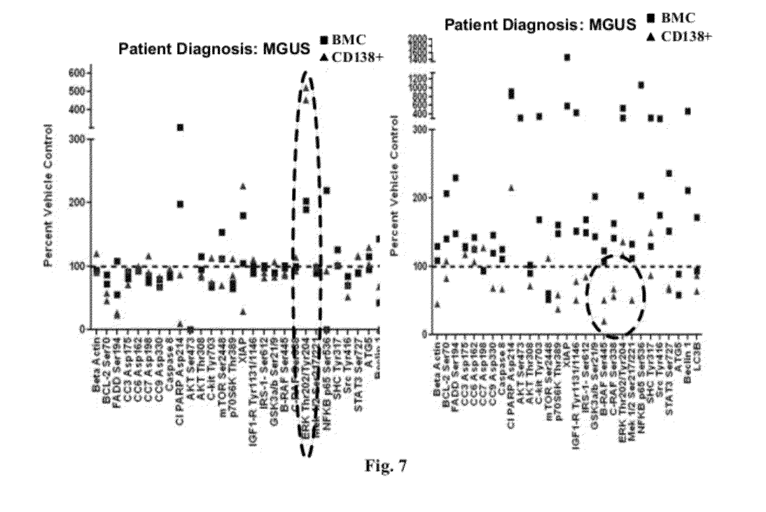

Bone marrow aspirates were treated with unique drug combinations of Chloroquine and HA14-1 (Bcl-2 inhibitor), Chloroquine and Rapamycin, or Chloroquine and tyrosine kinase inhibitors such as Sunitinib/Dasatinib/Lapatinib, etc., permitting the simultaneous evaluation of treatment effects on both myleoma (diseased) and non-diseased cells (FIG. 4). We were able to measure compensatory up-regulation of cell signaling pathways by reverse phase protein microarray as a prognostic indicator of drug resistance/drug response. In addition, this method allowed the differential effect of treatment on the CD138+ and non-CD138+ cell populations to be quantitated. This method may be used at least for determining toxicity on normal cells in individual patients for therapeutic decisions.

We propose at least one means of treating any stage of multiple myeloma (where the myeloma cells are growth inhibited, suppressed, or killed, to a greater extent compared to the non-myeloma cells) with the combination of an autophagy inhibitor with a non-chemotherapeutic agent (such as a tyrosine kinase inhibitor, small molecule inhibitor or a therapeutic antibody with examples listed in Table 1 below). We also propose the combination of an autophagy inhibitor with non-chemotherapeutic agents for the treatment of patients with myeloma pre-cursors diseases, Monoclonal Gammopathy, Multiple Gammopathy of Unknown origin Syndrome (MGUS), amyloidosis, plasmacytoma, or any other plasma cell related disease.

This work can quantitatively measure the phosphorylation, cleavage or total forms of kinases, phosphatases and other cell signaling proteins in bone marrow aspirate and bone marrow core samples for treatment regimen stratification. Specific inhibitors, such as gefitinib, erlotininb, and surafinib, or combinations of inhibitors with steroids (dexamethasone) and/or autophagy inhibitors can be tested ex vivo using a patient's bone marrow aspirate to predict which patient will respond to a particular therapy or combination. The multiplexed nature of the reverse phase protein microarray technology permits quantitative measurement of multiple cell signaling proteins. This work can be used to generate a functional multiple myeloma or leukemia classifier based on drug target activation and test the hypothesis that cell signaling activation portraits can predict a priori which targeted therapies will best cause cell death.

This work can provide simultaneous assessments of treatment effects on diseased and non-diseased cell populations. For example, Non-plasma cells and plasma (myeloma) cells can be concomitantly studied for therapeutic efficacy for an individual patient. Analysis of both diseased and non-diseased cell populations, under the same conditions, with the same treatments, can be used to predict potential toxicity as well as efficacy.

The biologic mechanisms involved in the pathogenesis of multiple myeloma (MM)-induced osteolytic bone disease are complex. Physiological interactions between the serotoninergic and skeletal systems are implicated by clinical observations [1]. The RPMA used in this invention has revealed a new role for serotonin signaling in myeloma/MGUS osteolytic bone disease.

We propose at least one means of individualizing treatment for bone disease including primary and metastatic neoplasia, or treating or preventing osteolytic bone disease which comprises a serotonin modulator alone, or in combination with, an autophagy inhibitor and/or a non-chemotherapeutic agent (examples listed in Table 1 below).

The monoamine serotonin [5-hydroxytryptamine (5-HT)] has previously been investigated as a neurotransmitter, synthesized by a two-step pathway in which tryptophan hydroxylase is the rate-limiting enzyme. Circulating 5-HT is principally stored in platelet-dense granules. Aggregated immunoglobulins derived from all the IgG subclasses, isolated from healthy controls or myeloma patients, induce platelet granules release in the absence of antigen or particulate matter, in a dose dependent manner [2].

The brainstem-derived serotonin (BDS) positively regulates bone mass following binding to 5-HT2C receptors on ventromedial hypothalamic neurons. This is opposed by platelet-derived serotonin (PDS) which induces bone lysis and osteoclast activation.

Immunoglobulins have been shown to induce platelet release a) when participating in immune reactions as antigen-antibody complexes or b) by non-immune mechanisms such as coating of glass or polymethylmethacrylate beads.

MM patients with evidence of osteolytic lesions exhibited an increase in the concentration of serum tryptophan and serotonin [3], while that of tyrosine, dopamine, and noradrenaline was decreased [3].

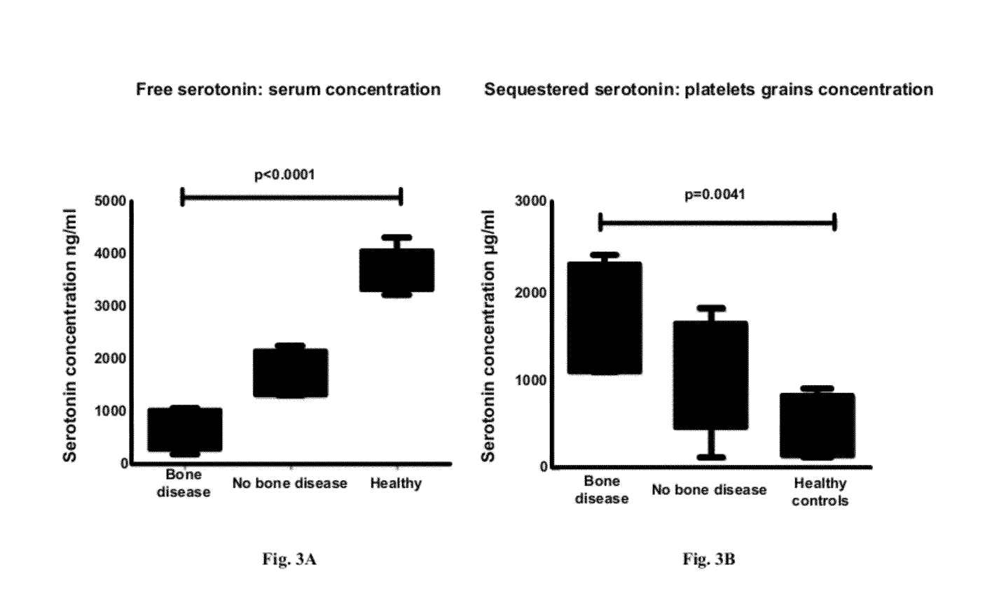

We found that bone marrow cells from patients with osteolytic multiple myeloma has higher levels of serotonin, RANK, Arrestin, DKK1 and TNF alpha compared to non-osteolytic myeloma patients (FIG. 3). Increased circulating-serotonin levels released from platelets by immunoglobulin complexes may alter the RANK/RANKL ratio in the BM environment and promote MM osteolytic lesion.

These data indicate that the 5-HT system plays an important role in bone homeostasis through effects on osteoclast function and that the serotonin system may be involved in the pathogenesis of MM-induced bone disease. Therefore serotonin regulation is a new therapeutic target for preventing or treating osteolytic bone disease associated with multiple myeloma or other conditions.

According to embodiments, bone disease may include preneoplastic lesions originating in bone marrow, bone metastasis, monoclonal gammopathy of unknown significance (MGUS), brittle bone disease, multiple myeloma, osteogenesis imperfecta (OI), osteolytic bone disease, amyloidosis, alterations of bone marrow hematopoietic precursor cells, and myelodysplasia.

According to additional embodiments, cells may include osteoclasts, osteoblasts, B cells, T cells, macrophages, megakaryocytes, bone marrow stroma stem cells, bone marrow stem cells, platelets, and white blood cells, blood vessel associated cells, hematopoetic precursor cells, and sympathetic and parasympathetic neuronal elements in the bone.

Chemical compounds contemplated may include 4-aminoquinolines well known to skilled artisans including chloroquine, Amodiaquine, quinine, and hydroxychloroquine.

In this specification, “a” and “an” and similar phrases are to be interpreted as “at least one” and “one or more”.

The disclosure of this patent document incorporates material which is subject to copyright protection. The copyright owner has no objection to the facsimile reproduction by anyone of the patent document or the patent disclosure, as it appears in the Patent and Trademark Office patent file or records, for the limited purposes required by law, but otherwise reserves all copyright rights whatsoever.

While various embodiments have been described above, it should be understood that they have been presented by way of example, and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. Thus, the present embodiments should not be limited by any of the above described exemplary embodiments. In particular, it should be noted that, for example purposes, the above explanation has focused on at least the example(s) serotonin modulators. However, one skilled in the art will recognize that embodiments of the invention could be serotonin agonists, serotonin antagonists, or both. Additionally, it should be noted that, for example purposes, the above explanation has focused on at least the example(s) chloroquine. However, one skilled in the art will recognize that embodiments of the invention could be 4-aminoquinolines other than chloroquine.

In addition, it should be understood that any figures which highlight the functionality and advantages, are presented for example purposes only. The disclosed method is sufficiently flexible and configurable, such that it may be utilized in ways other than that shown. For example, the steps listed in any figure may be re-ordered or only optionally used in some embodiments.

Further, the purpose of the Abstract of the Disclosure is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract of the Disclosure is not intended to be limiting as to the scope in any way.

Finally, it is the applicant's intent that only claims that include the express language “means for” or “step for” be interpreted under 35 U.S.C. 112, paragraph 6. Claims that do not expressly include the phrase “means for” or “step for” are not to be interpreted under 35 U.S.C. 112, paragraph 6.

### Example 1

### Treating Bone Marrow with 4-Amino Quinolines

Bone marrow aspirates were treated with unique drug combinations of Chloroquine and HA14-1 (Bcl-2 inhibitor), Chloroquine and Rapamycin, or Chloroquine and tyrosine kinase inhibitors such as Sunitinib/Dasatinib/Lapatinib, etc., permitting the simultaneous evaluation of treatment effects on both myeloma (diseased) and non-diseased cells (FIG. 4).

Compensatory up-regulation of cell signaling pathways was measured by reverse phase protein microarray as a prognostic indicator of drug resistance. In addition this method allowed the differential effect of treatment on the CD138+ and non-CD138+ cell populations to be quantified. This method may be used for determining toxicity on normal cells in individual patients for therapeutic decisions.

### Example 2

### Measuring the In Vivo State of the Pathway

In order to measure the in vivo state of the pathways bone marrow biopsies fixed with preservatives that stabilizes phosphoproteins and permits paraffin embedding without decalcification. Microdissected cellular subpopulations are by reverse phrase protein microarray (RPMA).

### Example 3

### Human Ex-Vivo Efficacy Testing of Serotonin Modulators in Multiple Myeloma Patients

Patient selection. A number of human patients were enrolled in the study. All Multiple Myeloma patients had measurable disease defined as a monoclonal component evaluated at serum electrophoresis of at least 1 g/dL of IgG, or 0.5 g/dL of IgA, or urinary excretion of at least 200 mg per day of a monoclonal light chain. All MGUS (monoclonal gammopathy of unknown significance) patients had stable chronic disease with at least two years of follow-up.

Samples. Samples were collected from patients with either newly diagnosed or relapsed multiple myeloma or MGUS. Bone marrow aspirates were collected by needle aspiration from the posterior iliac crest and placed immediately into sodium heparin vacutainer (without vacuum). For sera collection, blood was spun within two hours of collection at 1600×g for 10 min. at room temperature. Serum was removed and stored at −80° C. for a maximum of two months.

Platelets were isolated from EDTA anti-coagulated whole blood which was spun at 2000×g for 10 min. Platelet rich plasma was aspirated with a plastic pipette and transferred into a plastic tube. Platelets were quantified by manual counting in a hemocytometer. The platelet rich plasma was washed twice with physiological saline and centrifugation at 2000×g for 10 min., and then resuspended back to the original volume using distilled water. Platelets were stored at minus −80° C. for a maximum of two months.

Plasma cell isolation and protein extraction. CD 138+ cells were separated from the non-CD138+ bone marrow microenvironment cells via immunomagnetic sorting (Stem Cell Technologies human whole blood isolation kit) (Stem Cell Technologies, Vancouver, Canada). Cells were incubated with magnetic beads (Stem Cell Technologies) coated with antibodies against CD138 and positioned directly on top of a 48 well microtiter plate containing aligned neodymium magnets. In this step, the plasma cells attached to the beads remained on the bottom of the microtiter plate, while the bone marrow microenvironment elements (not expressing the CD138 marker) remained in the supernatant. The non-bound cells were removed by aspiration. The CD138+ cells were removed from the plate by removing the plate from the magnetic field and aspirating the cells.

Reverse protein microarray. 15 core biopsies obtained from other patients with multiple myeloma at different clinical stages were retrospectively examined for 20 differentiated bone proteins using reverse-phase protein microarray (RPMA). Core proteins were diluted in protein extraction buffer and denatured by heating for 5 min. at 100° C. prior to dilution of the microtiter plate. Serial two-fold dilutions of the lysates were printed in duplicate on glass backed nitrocellulose array slides (Schott) in a dilution curve representing undiluted lysate, 1:2, 1:4, and negative control dilutions, using an Aushon 2470 arrayer equipped with 350 μm pins. Each spot was printed with approximately 30.0 nl of the lysate per spot. Slides were stored with desiccant (Drierite, W. A. Hammond, Xenia, Ohio) (minus 20° C. prior to immunostaining. Immunostaining was performed on an automated slide stainer according to the manufacturer's instructions (Autostainer catalyzed signal amplification (CSA), Dako, Carpinteria, Calif.). Each slide was incubated with a single primary antibody at room temperature. Each array was probed with a single polyclonal or monoclonal primary antibody selected from the validated antibodies applied to the reverse phase protein arrays (see Table 2).

The negative slide was incubated with antibody diluent. A secondary antibody used was goat anti-rabbit IgG heavy plus chain (1:10,000) (Vector Laboratories, Burlingame, Calif.). Subsequent protein detection was amplified via horseradish peroxidase-mediated biotinyl tyramide with chromogenic detection (Diaminobenzidine) according the manufacturer's instructions (Dako). Total protein per microarray spot was determined with Sypro® Ruby protein blot stain (Invitrogen). Single-stranded DNA (ssDNA) was, quantified on an RPMA using an antibody directed against ssDNA. Arrays were scanned at 600 dpi on a flatbed scanner (UMAX power look 2000), spot intensity was analyzed with commercial image software ImageQuant v.5.0, data was normalized, and a standardized single value was generated for each sample on the array.

Serotonin detection in peripheral blood. Serotonin levels in both sera and platelets were detected using a commercial competitive binding enzyme immunoassay kit (serotonin EIA kit, catalog #900-175), according to the manufacturer's procedures. Briefly, controls were prepared according to the manufacturer's protocol. Sera were diluted 1:10 and platelets 1:2 in duplicate.

Detection of plasma cell serotonin using multiple myeloma cell line model. RPMI-8226 and U266 myeloma cell lines were obtained from the American Type Culture Collection (ATCC, Manassas, Va.) and cultured in RPMI 1640 medium (Invitrogen) with addition of 10% FBS without antibiotics for 24, 48 and 72 hours in duplicate. Cell supernatants and cell pellets were collected at each time point. Cells were counted and extracted in extraction buffer at a concentration of 1,000,000 cells per 50 μl. Medium alone and extraction buffer were used as baseline controls. Serotonin content was measured using 2 different assays: IBL Serotonin ELISA kit and Reverse Phase Protein Microarray (RPMA). Newly synthesized serotonin in supernatant samples was defined as the difference between serotonin content in the sample and serotonin content in conditioned medium alone. Newly synthesized serotonin in cell samples was defined as the difference in serotonin content between the sample and the extraction buffer. Serotonin content in supernatants and cells was measured using the IBL Serotonin ELISA kit according to manufacturer's directions. Reverse-phase Protein Microarray: Supernatants and cell samples were printed in a 4-point 2-fold dilution curve in duplicate on the same array. One slide was probed with an anti-Serotonin antibody (Invitrogen) and stained using an HRP immunostaining system coupled with a DAB colorimetric dye. The second slide was probed with only antibody diluent in place of the primary antibody, and used as a negative control. Increased levels of serotonin over time were noted in the conditioned medium from the myeloma cell lines.

Determination of bone disease in Multiple Myeloma patients. Skeletal radiologic surveys were examined and evaluated for the presence of lytic lesions using the score previously described by Noonan et al. (Noonan, et al, 2010). Briefly, a score of 0 was assigned to patients with no evidence of lytic bone disease, 1 to patients with one lesion less than 1 cm in diameter, 2 to patients with one lesion greater than 1 cm in diameter, 3 to patients with multiple lesions each less than 1 cm in diameter, and 4 to patients with multiple lesions greater than 1 cm in diameter. Patients with lytic bone disease were found to have a higher content of serotonin associated with the bone marrow cell aspirate compared to the patients without active bone disease.

### Example 4

### Treating Human Bone Disease In Vivo with Serotonin Modulators

In order to treat subjects with bone disease, subjects are treated with a serotonin modulator in an effective amount. By way of nonlimiting example, PROZAC® (fluoxetine capsules, USP) is a selective serotonin reuptake inhibitor for oral administration. It is also marketed for the treatment of premenstrual dysphoric disorder (Sarafem®, fluoxetine hydrochloride). It is designated (RMG)-N-methyl-3-phenyl-3-[(α,α,α-trifluoro-p-tolyl)oxy] propylamine hydrochloride and has the empirical formula of C17H18F₃NO.HCl. Its molecular weight is 345.79. Each Pulvule® contains fluoxetine hydrochloride equivalent to 10 mg (32.3 μl), 20 mg (64.7 μl), or 40 mg (129.3 μl) of fluoxetine. PROZAC WEEKLY™ capsules contain enteric-coated pellets of fluoxetine hydrochloride equivalent to 90 mg (291 μl) of fluoxetine. The Prozac dosage range for the treatment of bone disease may include from about 20 mg to about 80 mg per day. The Prozac weekly dosage for the treatment of bone disease may include about 90 mg (291 μmol) of fluoxetine administered once a week.

### Example 5

### Treating Human Bone Disease In Vivo with Serotonin Modulators and Other Agents

In order to treat subjects with bone disease, subjects are treated with a combination of a serotonin modulator and an autophagy pathway modulator. Serotonin modulators are known to skilled artisans as set forth hereinabove. By way of non-limiting example, subjects are treated with a serotonin modulator and chloroquine. For example, PROZAC® (fluoxetine capsules, USP) administration includes dosing as set forth hereinabove. Furthermore, the Prozac dosage range for the treatment of bone disease includes from about 20 mg to about 80 mg per day. The PROZAC WEEKLY™ dosage for the treatment of bone disease includes about 90 mg (291 μl) of fluoxetine administered once a week.

Chloroquine phosphate, USP is a 4-aminoquinoline compound for oral administration. Chloroquine phosphate, USP, a 4-aminoquinoline, is an antimalarial and amebicidal drug that inhibits autophagy by preventing the fusion of the autophagosome with the lysosome. Chemically, it is 7-chloro-4-[[4-(diethylamino)-1-methylbutyl]amino]quinoline phosphate (1:2) (C18H26CIN3.2H3P04; Molecular Weight: 515.86).

Chloroquine tablets may include tablets that contain 500 mg of Chloroquine phosphate USP, equivalent to 300 mg Chloroquine base. Chloroquine tablets for the treatment of bone disease may include tablets that contain about 500 mg of Chloroquine phosphate USP, equivalent to about 300 mg Chloroquine base.

A Chloroquine phosphate dosage is often expressed in terms of equivalent Chloroquine base. Each 500 mg tablet of Chloroquine phosphate tablets, USP contains the equivalent of 300 mg. The dosage is preferably calculated by body weight. 10 mg base per kg (but not exceeding a single dose of 600 mg base). A typical adult dose for the treatment of bone disease may include a dose of about 500 mg (=300 mg base) on exactly the same day of each week.

### Example 6

### Treating Human Bone Disease In Vivo with 4-Amino Quinolines, Tyrosine Kinase Inhibitors, and Bisphosphonates

This non-limiting prospective example demonstrates how neoplastic and/or preneoplastic bone disease may be treated with a combination of agents.

Patients with neoplastic and/or preneoplastic bone disease are treated with 4-amino quinolines (for example for chloroquine), tyrosine kinase inhibitors and bisphosphonates. Patients are administered a three drug cocktail comprising chloroquine, Prozac, and a bisphosphonate.

Examples of bisphosphonates are well known to skilled artisans and include Clodronate. Clodronate may be administered orally in a dosage of about 1000 to about 4000 mg a day in at least one or two doses. Clodronate may also be administered orally in a dosage of about 1600 mg to about 2400 mg per day.

Clodronate may also be administered by intravenous infusion at a dosage of about 300 mg over at least two hours once a day as needed.

Treatment with 4-amino quinolines, tyrosine kinase inhibitors, and bisphosphonates, may also include a step of selecting patients with at least one of an altered autophagy pathway, brittle bone disease, multiple myeloma, osteogenesis imperfecta, osteolytic bone disease, amyloidosis, monoclonal gammopathy, MGUS, alterations in bone marrow hematopoetic precursor cells, and myelodysplasia.

### Example 7

### Treating Human Bone Disease with 4-Amino Quinolines, Tyrosine Kinase Inhibitors, and Monoamine Oxidase Inhibitors

This non-limiting prospective example demonstrates how neoplastic and/or preneoplastic bone disease may be treated with a combination of agents.

Patients with neoplastic and/or preneoplastic bone disease are treated with 4-amino quinolines (for example for chloroquine), tyrosine kinase inhibitors and monoamine oxidase inhibitors (for example Phenelzine sulfate). Examples of monoamine oxidase inhibitors are well known to skilled artisans and may include Phenelzine Sulfate.

By way of nonlimiting example, patients are administered at least three drugs including chloroquine or alternatively another 4-aminoquinoline, Prozac or alternatively another serotonin modulator, and a Phenelzine sulfate or alternatively another monoamine oxidase inhibitor. The dosing for chloroquine and tyrosine kinase inhibitors for the treatment of bone disease is set forth hereinabove. For the treatment of bone disease in a subject, Phenelzine sulfate may be administered orally with a dosage of about 15-90 mg daily and in keeping with the knowledge of those skilled in the art regarding the risks associated with monoamine oxidase inhibitors.

Treatment with 4-amino quinolines, tyrosine kinase inhibitors, and monoamine oxidase inhibitors, may also include a step of selecting patients with at least one of an altered autophagy pathway, brittle bone disease, multiple myeloma, osteogenesis imperfecta, osteolytic bone disease, amyloidosis, monoclonal gammopathy, MGUS, alterations in bone marrow hematopoetic precursor cells, and myelodysplasia.

TABLE 1
Autophagy combination therapy example agents.

Inhibitors
17-DMAG
8-hydroxy Guanosine
AKT Inhibitor IV
AKT inhibitor X
AKT inhibitor XI
AMPK Inhibitor, Compound C
BAY 11-7082
Bcr-abl Inhibitor
Bortezomib
Carfilzomib
Caspase-3 Inhibitor VII
Caspase-8 inhibitor I
Caspase-9 inhibitor II
CGP041251 (Midostaurin)
Chloroquine
Cox II Inhibitor
Dasatinib
Dexamethasone
EGFR inhibitor II, BIBX1382
EGFR/Erb-2/Erb-4 Inhibitor
ERK inhibitor II, Negative control
ERK inhibitor III
erlotinib
FGF/VEGF Receptor Tyrosine Kinase Inhibitor, PD173074
Gefitinib
Glycogen Phosphorylase Inhibitor
Granzyme B inhibitor I
HA14-1
HNMPA-(AM)₃ (Insulin Receptor TKI inhibitor)
Honokoil
HSP90 Inhibitor
IGF-1R Inhibitor II
IGF-1R PPP
Imatinib
Imatinib
Jak2 Inhibitor II
Jak3 Inhibitor I
JNK Inhibitor I, (L)-Form
K2529
Lapatinib
LY294002
MAPK Inhibitor PD169316
Mek 1& 2 inhibitor SL327
Melatonin
Melphalan
NVP-BEZ235
NVP-Raf-265
NVP-LBH589
NVP-AMN107 (Nilotinib)
NVP-TKI258-CU-2
PARP Inhibitor XI, DR2313
PD153035 (EGFR Inhibitor)
PD98059 (MEK inhibitor)
PDGF Receptor Tyrosine Kinase Inhibitor I
PI 3-Kα Inhibitor IV
PI 3-Kγ Inhibitor II
Proteasome Inhibitor IX, AM114
RAD001
Rapamycin
Resveratrol
Sorafinib
Src Kinase Inhibitor II
Sunitinib
Terphenyl (FWF416)
VEGF Receptor Tyrosine Kinase Inhibitor III, KRN633
Wortmannin
ZM 336372 (c-Raf inhibitor)
hydroxychloroquine
3-methyladenie
clomipramine
ethyl pyruvate
glycyrrhizin
Asparagine (Asn)
Leupeptin
Serotonin or serotonin related inhibitor
Serotonin modulator agents such as serotonin reuptake inhibitors, or
serotonin receptor antagonists
Bisphosphonates and other nitrogenous or non-nitrogenous
inhibitors such as Clodronate or Zoledronic Acid
Collagenase inhibitors such as Matrix Metalloproteinase Inhibitors

TABLE 2
List of antibodies to e proteins that can be evaluated by reverse phase
protein microarray.
Post-translationally Modified Proteins

| Protein/antibody | MW (kDa) |
|---|---|
| phospho-Tyrosine (P-Y-100) | NA |
| 4E-BP1 (S65) | 15-20 |
| 4E-BP1 (T37/46) | 15-20 |
| 4E-BP1 (T70) | 15-20 |
| 4G10 (anti Phosphotyrosine) | many |
| c-Abl (T735) | 120 |
| c-Abl (Y245) | 135 |
| Acetyl-CoA Carboxylase (S79) | 280 |
| Ack1 (Y284) | 135 |
| Ack1 (Y857/858) | 135 |
| Adducin (S662) | 80, 120, |
| | 110 |
| Akt (S473) | 60 |
| Akt (S473) | 60 |
| Akt1/PKB alpha (S473) (SK703) | 60 |
| Akt (T308) | 60 |
| ALK (Y1586) | 80, 220 |
| AMPKalpha1 (S485) | 62 |
| AMPKBeta1 (S108) | 38 |
| Arrestin1 (Beta) (S412) (6-24) | 50 |
| ASK1 (S83) | 155 |
| ATF-2 (T71) | 70 |
| ATF-2 (T69/71) | 70 |
| ATP-Citrate Lyase (S454) | 125 |
| Aurora A (T288)/B (T232)/C (T198) | 35, 40, 48 |
| (D13A11) | |
| Bad (S112) | 23 |
| Bad (S136) | |
| Bad (S155) | |
| Bcl-2 (S70) (5H2) | 28 |
| Bcl-2 (T56) | 28 |
| Bcr (Y177) | 160, 210 |
| Caspase-3, cleaved (D175) | 17, 19 |
| Caspase-3, cleaved (D175) (5A1) | 17, 19 |
| Caspase-6, cleaved (D162) | 18 |
| Caspase-7, cleaved (D198) | 20 |
| Caspase-9, cleaved (D315) | 35 |
| Caspase-9, cleaved (D330) | 17, 37 |
| Catenin (beta) (S33/37/T41) | 85 |
| Catenin (beta) (T41/S45) | 85 |
| Chk1 (S345) | 56 |
| Chk2 (S33/35) | 62 |
| Cofilin (S3) (77G2) | 19 |
| CREB (S133) | 43 |
| CREB (S133) (1B6) | 43 |
| CrkII (Y221) | 42 |
| CrkL (Y207) | 39 |
| Cyclin D1 (T286) | 36 |
| EGFR (S1046/1047) | 175 |
| EGFR (Y845) | 175 |
| EGFR (Y992) | 175 |
| EGFR (Y1045) | 175 |
| EGFR (Y1068) | 175 |
| EGFR (Y1068) (1H12) | 175 |
| EGFR (Y1148) | 175 |
| EGFR (Y1148) | 185 |
| EGFR (Y1173) | 175 |
| EGFR (Y1173) (9H2) | 175 |
| EGFR (Y1173) (53A3) | 175 |
| eIF2alpha (S51) (119A11) | 40 |
| eIF4E (S209) | 25 |
| eIF4G (S1108) | 200 |
| Elk-1 (S383) | 62 |
| eNOS (S113) | 140 |
| eNOS (S1177) | 140 |
| eNOS/NOS III (S116) | 132 |
| ErbB2/HER2 (Y877) | 185 |
| ErbB2/HER2 (Y1248) | 185 |
| ErbB2/HER2 (Y1248) | 185 |
| ErbB2/HER2 (Y1248) | 185 |
| ErbB3/HER3 (Y1197) (C56E4) | 185 |
| ErbB3/HER3 (Y1289) (21D3) | 185 |
| ERK ½ (T202/Y204) | 42, 44 |
| Estrogen Receptor alpha (S118) | 66 |
| Estrogen Receptor alpha (S118) (16J4) | 66 |
| Etk (Y40) | 76 |
| Ezrin (Y353) | 80 |
| Ezrin (T567)/Radixin (T564)/Moesin (T558) | 75, 80 |
| FADD (S194) | 28 |
| FAK (Y397) (18) | 125 |
| FAK (Y576/577) | 125 |
| FKHR (S256) | 82 |
| FKHRL1 (S253) | 100 |
| FKHR (T24)/FKHRL1 (T32) | 68, 97 |
| alpha-Fodrin, cleaved (D1185) | 150 |
| FRS2-alpha (Y436) | 80-85 |
| Gab1 (Y627) | 110 |
| GSK-3alpha (S21) (46H12) | 51 |
| GSK-3alpha/beta (S21/9) | 46, 51 |
| GSK-3alpha (Y279)/beta (Y216) | 47, 51 |
| GSK-3beta (S9) | 46 |
| Histone H3 (S10) Mitosis Marker | 17 |
| Histone H3 (S28) | 17 |
| HSP27 (S82) | 27 |
| HSP90a (T5/7) | 90 |
| IGF-1 Rec (Y1131)/Insulin Rec (Y1146) | 90 |
| IGF-1R (Y1135/36)/IR (Y1150/51) (19H7) | 90 |
| IkappaB-alpha (S32/36) (5A5) | 40 |
| IkappaB-alpha (S32/36) (39A1431) | 42 |
| IRS-1 (S612) | 180 |
| Jak1 (Y1022/1023) | 130 |
| Jak2 (Y1007/1008) | 125 |
| c-Kit (Y703) (D12E12) | 145 |
| c-Kit (Y719) | 120, 145 |
| Lamin A, cleaved (D230) | 45, 50 |
| Lck (Y505) | 56 |
| LIMK1 (T508)/LIMK2 (T505) | 72 |
| LKB1 (S334) | N/A |
| LKB1 (S428) | N/A |
| MAPK (pTEpY) | 42, 44 |
| MARCKS (S152/156) | 80, 87 |
| MEK1 (S298) | 45 |
| MEK½ (S217/221) | 45 |
| Met (Y1234/1235) | 145 |
| MSK1 (S360) | 90 |
| Mst1 (T183)/Mst2 (T180) | 59 |
| mTOR (S2448) | 289 |
| mTOR (S2481) | 289 |
| NF-kappaB p65 (S536) | 65 |
| NPM (T199) | 38 |
| p27 (T187) | 27 |
| p38 MAP Kinase (T180/Y182) | 40 |
| p40 phox (T154) | 40 |
| p53 (S15) | 53 |
| p70 S6 Kinase (S371) | 70, 85 |
| p70 S6 Kinase (T389) | 70, 85 |
| p70 S6 Kinase (T412) | 70 |
| p90RSK (S380) | 90 |
| PAK1 (S199/204)/PAK2 (S192/197) | 61-67, 68-74 |
| PAK1 (T423)/PAK2 (T402) | 61-67, 68-74 |
| PARP, cleaved (D214) | 89 |
| Paxillin (Y118) | 68 |
| PDGF Receptor alpha (Y754) (23B2) | 198 |
| PDGF Receptor beta (Y716) | 190 |
| PDGF Receptor beta (Y751) | 190 |
| PDK1 (S241) | 63 |
| PKC alpha (S657) | 82 |
| PKC alpha/beta II (T638/641) | 80, 82 |
| PKC (pan) (betaII S660) | 78, 80, 82, 85 |
| PKC delta (T505) | 78 |
| PKC theta (T538) | 79 |
| PKC zeta/lambda (T410/403) | 76 |
| cPLA2 (S505) | 110 |
| PLCgamma1 (Y783) | 155 |
| PLK1 (T210) | 68 |
| PRAS40 (T246) | 40 |
| PRK1 (T774)/PRK2 (T816) | 120, 140 |
| Progesterone Receptor (S190) | 90, 118 |
| PTEN (S380) | 54 |
| Pyk2 (Y402) | 116 |
| Raf (S259) | 74 |
| A-Raf (S299) | 68 |
| B-Raf (S445) | 95 |
| c-Raf (S338) (56A6) | 74 |
| Ras-GRF1 (S916) | 155 |
| Rb (S780) | 110 |
| Ret (Y905) | 175 |
| RSK3 (T356/S360) | 90 |
| S6 Ribosomal Protein (S235/236) (2F9) | 32 |
| S6 Ribosomal Protein (S240/244) | 32 |
| SAPK/JNK (T183/Y185) | 46, 54 |
| SEK1/MKK4 (S80) | 44 |
| SGK (S78) (D36D11) | 54 |
| Shc (Y317) | 46, 52, 67 |
| SHIP1 (Y1020) | 145 |
| SHP2 (Y580) | 70 |
| Smad1 (S/S)/Smad5 (S/S)/Smad8 (S/S) | 60 |
| Smad2 (S465/467) | 58 |
| Smad2 (S245/250/255) | 60 |
| Src Family (Y416) | 60 |
| Src (Y527) | 60 |
| Stat1 (Y701) | 84, 91 |
| Stat1 (Y701) | 92 |
| Stat2 (Y690) | 113 |
| Stat3 (S727) | 79, 86 |
| Stat3 (Y705) (9E12) | 92 |
| Stat3 (Y705) (D3A7) | 79, 86 |
| Stat4 (Y693) | 81 |
| Stat5 (Y694) | 90 |
| Stat6 (Y641) | 110 |
| Syk (Y525/526) | 72 |
| Tuberin/TSC2 (Y1571) | 200 |
| Tyk2 (Y1054/1055) | 140 |
| VASP (S157) | 51 |
| Vav3 (Y173) | 95 |
| VEGFR 2 (Y951) | 230 |
| VEGFR 2 (Y996) | 230 |
| VEGFR 2 (Y1175) (19A10) | 230 |
| Zap-70 (Y319)/Syk (Y352) | 70, 72 |
| 14-3-3 zeta, gamma, eta | 27 |
| 4E-BP1 | 15-20 |
| Abl SH2 domain | 140, 210 |
| Actin, Beta | 45 |
| Akt | 60 |
| Akt2 (5B5) | 60 |
| Albumin | 67 |
| Aldehyde Dehydrogenase 1 | 55 |
| Aldehyde Dehydrogenase (ALDH) | 55 |
| Aldehyde Dehydrogenase 2 (ALDH2) | 56 |
| Androgen Receptor | 110 |
| Annexin I | 38 |
| Annexin II | 36 |
| ANT (N-19) | 33 |
| Apaf-1 | 130, 140 |
| APC2 Ab-1 | 92 |
| Arrestin ½, Beta | 50 |
| Atg5 | 55 |
| Atg12 | 16, 53 |
| Aurora A/AIK | 48 |
| Axin1 (C76H11) | 110 |
| Bad | 23 |
| Bak | 25 |
| Bax | 20 |
| Bcl-2 | 28 |
| Bcl-xL | 30 |
| Beclin-1 | 60 |
| Beclin-1 | 60 |
| Biliverdin Reductase (BVR) | 33/41-42 |
| BLVRB (biliverdin reductase B) (2F4) | 37 |
| Bmi-1 (10C7.2) | ~33 |
| Bub3 | 40 |
| E-Cadherin | 135 |
| N-Cadherin | 140 |
| Calreticulin (FMC 75) | 48 |
| CaM Kinase II | 50 |
| Caspase-3 | 17, 19, 35 |
| Caspase-7 | 20, 35 |
| Caspase-8 (1C12) | 18, 43, 57 |
| Caspase-8 | 54, 55 |
| Caspase-9 | 17, 35, 37, 47 |
| Catenin(beta) | 92 |
| Cathepsin B (G60) | 39-42 |
| CCT5 (Chaperonin Containing TCP1, | 60 |
| subunit 5) | |
| CD3 epsilon | 20-25 |
| CD3 zeta (1D4) | 21 |
| CD3 zeta (8D3) | 16, 32 |
| CD5L | 38 |
| CD9 (C-4) | 24 |
| CD24 (FL-80) | 45 |
| CD24 (GPI-linked surface mucin) Ab-2 | 30-70 |
| (SN3b) | |
| CD44 (156-3C11) | 80 |
| CD45 (BRA-55) | 180-240 |
| CD45 | 180-220 |
| CD63 (MX-49.129.5) | 53 |
| CD133 (W6B3C1) | 120 |
| CDK2 (78B2) | 33 |
| Cofilin (D59) | 19 |
| Collagen Type I (NFI/20) | 70-90 |
| Complement factor H | 150 |
| Cox-2 (33) | 70 |
| CREB | 43 |
| Cripto | 18, 20 |
| Crystallin, alpha/Beta | 20 |
| Cu/Zn Superoxide Dismutase (SOD) | 19/23 |
| Cyclin A (BF683) | 55 |
| Cyclin B1 (V152) | 60 |
| Cyclin D1 (G124-326) | 36 |
| Cyclin D1 (DCS6) | 36 |
| Cytochrome Vb | 13.7 |
| Cytochrome C | 14 |
| Cytochrome C oxidoreductase | 19.6 |
| Cytokeratin 8 | 54 |
| DEPTOR | 46 |
| Destrin | 19 |
| DGK | 83 |
| DKK1 | 30, 35 |
| Dvl2 (30D2) | 90-95 |
| Dvl3 | 88-93 |
| EGFR | 175 |
| EGFR (L858R Mut-Spec) | 175 |
| Egr1 | 85 |
| elF4G | 220 |
| eNOS | 140 |
| ErbB2/HER2 | 185 |
| ErbB2/HER2 (44E7) | 185 |
| c-ErbB2/HER2 | 185 |
| c-ErbB2 (cytoplasmic domain) (N3/D10) | 185 |
| c-ErbB2/HER2 P185 (e2-4001) | 185 |
| ErbB3/HER3 (1B2) | 185 |
| ErbB4/HER4 (111B2) | 180 |
| ERK ½ | 42, 44 |
| ERK ½ (3A7) | 42, 44 |
| Estrogen Rec alpha (62A3) | 66 |
| Estrogen Rec alpha (1D5) | 66 |
| FAK | 116 |
| FHL2 | 44.6 |
| Fibronectin (IST-9) | 44, 47, 52 |
| Flg (H-76) | 110 |
| GFAP | 50 |
| GRB2 | 25 |
| GSK-3beta | 46 |
| HBB (Hemoglobin, beta) | 16 |
| Heme-Oxygenase-1 | 32 |
| Heparanase 1 | 65 |
| HIF-1alpha (54) | 120 |
| Histone Deacetylase 1 (HDAC1) | 62 |
| Histone Deacetylase 3 (HDAC3) | 49 |
| Histone Deacetylase 4 (HDAC4) | 140 |
| Histone H3, Di-Methyl (Lys9) | 15 |
| Histone H3, Di-Methyl (Lys27) | 15 |
| Histone H3, Pan-Methyl (Lys9) | 15 |
| HSP27 Protein 1 | 27 |
| HSP70 (C92F3A-5) | 70 |
| HSP90 (E289) | 90 |
| Ig Light Chain, Kappa | 25 |
| IGF-1 Receptor beta | 98 |
| IGF1 | 20 |
| IGFBP7 | 31 |
| IkappaB-alpha | 41 |
| IL-6 | 21-28 |
| IL-8 | 11 |
| IL-10 | 21 |
| IL-11 (H-169) | 23 |
| iNOS | 130 |
| Insulin Receptor beta (4B8) | 95 |
| IRS-1 | 180 |
| c-Kit (CD117) | 145, 125, 95 |
| LAMP-2 (H4HB4) | 120 |
| LC3B | 14, 16 |
| LC3B (D11) XP | 14, 16 |
| Lck | 56 |
| LDHA | 37 |
| LEDGF (26) | 52/75 |
| Lipocalin-1 (H-45) | 20 |
| LRP6 (C5C7) | 180, 210 |
| Lysine 48-Linkage Specific Polyubiquitin | multiple |
| MARCKS (2F12) | ≈60 |
| MEK½ | 45 |
| MGMT | 21 |
| MHC class I (F-3) | 46 |
| Microglobulin, beta-2 (FL-119) | 12 |
| MMP-9 | 84, 92 |
| MMP-11 | 55 |
| MMP-14 | 54, 66 |
| Mn Superoxide Dismutase (SOD) | 25 |
| MRPL11 (D68F2) | 21 |
| mTOR | 289 |
| Musashi | 35 |
| c-Myc | 57-70 |
| Naked1 (C30F10) | 59, 61 |
| Naked2 (C67C4) | 59, 61 |
| Nanog | 42 |
| NEDD8 | 9 |
| NF-kappaB | 75 |
| Nodal (5C3) | 42 |
| Notch 1 | 130/300 |
| Nucleobindin 1 Precursor | 54 |
| NUMB | 70 |
| Osteopontin (OPN) | 53 |
| p16 INK4A | 16 |
| Kip1/p27 (57) | 27 |
| p38 MAP Kinase | 40 |
| p53 | 53 |
| p62/SQSTM1 (5F2) | 62 |
| p70 S6 Kinase | 70, 85 |
| PAK2 | 61 |
| PCAF (C14G9) | 93 |
| PDGF Receptor beta | 190 |
| PDGF Receptor Beta (2B3) | 190 |
| PDGF Receptor beta (28E1) | 190 |
| PEDF | 50 |
| PHD-2/Egln1 | 50 |
| PI3-Kinase | 85 |
| PI3-Kinase p110gamma | 110 |
| PIAS1 | 76 |
| PKC alpha (M4) | 82 |
| PLC-gamma-1 | 155 |
| PLK1 | 62 |
| PP2A A Subunit | 62 |
| PP2A B Subunit | 62 |
| PRMT4/CARM1 | 63 |
| Proteasome 20S C2 | 30 |
| Protein Phosphatase 1 Beta (EP1804Y) | 37 |
| PSA (ER-PR8) | 33 |
| PSA (ER-PR8) | 33 |
| PTEN | 54 |
| RANK (H-300) | 90 |
| RANKL | 28, 36 |
| Ras-GRF1 | 155 |
| Ribosomal Protein L13a | 23 |
| RPE65 (H-85) | 65 |
| S100A7 calcium binding protein | 36.85 |
| SAPK/JNK | 46, 54 |
| SDF1Beta | 8.5 |
| Serotonin | 175 |
| SGK1 | 31, 50 |
| Skp1 | 19 |
| Smac/Diablo | 21 |
| Snail (SN9H2) | 29 |
| SOCS3 | 26 |
| Sox2 | 35 |
| c-Src (SRC 2) | 60 |
| ST6GALNAC5 | 38 |
| Stat1 | 84, 91 |
| Stat3 | 79, 86 |
| Stat5 (3H7) | 90 |
| Stat6 | 110 |
| SUMO-1 | many |
| SUMO-2/3 (18H8) | many |
| Survivin (71G4) | 16 |
| Syndecan-1 (CD138) | 90 |
| Synuclein, a/B (Syn205) | 18 |
| TGF-Beta (56E4) | 12, 45-60 |
| TIMP2 | 31 |
| TNF alpha | 17 |
| TNF-R1 (C25C1) | 55 |
| TOPK/PKB | 40 |
| Tubulin, alpha (B-5-1-2) | 50 |
| Tubulin, a/B | 55 |
| UBC3 | 32 |
| Ubiquitin (P4D1) | many |
| VDAC1 (N-18) | 30-35 |
| VEGF Receptor 2 (55B11) | 210, 230 |
| VHL | 24 |
| Vimentin | 57, 50 |
| Wnt5a/B (C27E8) | 45 |
| XIAP Antibody | 53 |
| ZAP-70 (2F3.2) | 70 |

The following references are included to provide background information as an aid to explain the embodiments:

### REFERENCES

1. Rosen, Nature Medicine (2009), 15:2, 145-6
2. Zimmermann, The Journal of Clinical Investigation (1975), 56, 828-834
3. Kurup, International Journal of Neuroscience, (2003), 113:9, 1221-1240.
4. Anderson, L and Seilhamer, J, A comparison of selected mRNA and protein abundances in human liver. Electrophoresis, 1997. 18(3-4): p. 533-7.
5. Araujo, R P and Liotta, L A, A control theoretic paradigm for cell signaling networks: a simple complexity for a sensitive robustness. Curr Opin Chem Biol, 2006. 10(1): p. 81-7.
6. Araujo, R P, Liotta, L A, and Petricoin, E F, Proteins, drug targets and the mechanisms they control: the simple truth about complex networks. Nat Rev Drug Discov, 2007. 6(11): p. 871-80.
7. Araujo, R P, Petricoin, E F, and Liotta, L A, A mathematical model of combination therapy using the EGFR signaling network. Biosystems, 2005. 80(1): p. 57-69.
8. Araujo, R P, Petricoin, E F, and Liotta, L A, Critical dependence of blood-borne biomarker concentrations on the half-lives of their carrier proteins. J Theor Biol, 2008. 253(3): p. 616-22.
9. Baudry, A, Bitard, J, Mouillet-Richard, S, Locker, M, Poliard, A, Launay, J M, and Kellermann, O, Serotonergic 5-HT(2B) receptor controls tissue-nonspecific alkaline phosphatase activity in osteoblasts via eicosanoids and phosphatidylinositol-specific phospholipase C. J Biol Chem, 2010. 285(34): p. 26066-73.
10. Bezieau, S, Devilder, M C, Avet-Loiseau, H, Mellerin, M P, Puthier, D, Pennarun, E, Rapp, M J, Harousseau, J L, Moisan, J P, and Bataille, R, High incidence of N and K-Ras activating mutations in multiple myeloma and primary plasma cell leukemia at diagnosis. Hum Mutat, 2001. 18(3): p. 212-24.
11. Bjork, K, Sjogren, B, and Svenningsson, P, Regulation of serotonin receptor function in the nervous system by lipid rafts and adaptor proteins. Exp Cell Res, 2010. 316(8): p. 1351-6.
12. Cao, W, Luttrell, L M, Medvedev, A V, Pierce, K L, Daniel, K W, Dixon, T M, Lefkowitz, R J, and Collins, S, Direct binding of activated c-Src to the beta 3-adrenergic receptor is required for MAP kinase activation. J Biol Chem, 2000. 275(49): p. 38131-4.
13. Chatterjee, M, Honemann, D, Lentzsch, S, Bommert, K, Sers, C, Herrmann, P, Mathas, S, Dorken, B, and Bargou, R C, In the presence of bone marrow stromal cells human multiple myeloma cells become independent of the IL-6/gp130/STAT3 pathway. Blood, 2002. 100(9): p. 3311-8.
14. Colucci, S, Brunetti, G, Rizzi, R, Zonno, A, Mori, G, Colaianni, G, Del Prete, D, Faccio, R, Liso, A, Capalbo, S, Liso, V, Zallone, A, and Grano, M, T cells support osteoclastogenesis in an in vitro model derived from human multiple myeloma bone disease: the role of the OPG/TRAIL interaction. Blood, 2004. 104(12): p. 3722-30.
15. Di Raimondo, F, Azzaro, M P, Palumbo, G, Bagnato, S, Giustolisi, G, Floridia, P, Sortino, G, and Giustolisi, R, Angiogenic factors in multiple myeloma: higher levels in bone marrow than in peripheral blood. Haematologica, 2000. 85(8): p. 800-5.
16. Disanza, A, Frittoli, E, Palamidessi, A, and Scita, G, Endocytosis and spatial restriction of cell signaling. Mol Oncol, 2009. 3(4): p. 280-96.
17. Durk, T, Panther, E, Muller, T, Sorichter, S, Ferrari, D, Pizzirani, C, Di Virgilio, F, Myrtek, D, Norgauer, J, and Idzko, M, 5-Hydroxytryptamine modulates cytokine and chemokine production in LPS-primed human monocytes via stimulation of different 5-HTR subtypes. Int Immunol, 2005. 17(5): p. 599-606.
18. Emmert-Buck, M R, Bonner, R F, Smith, P D, Chuaqui, R F, Zhuang, Z, Goldstein, S R, Weiss, R A, and Liotta, L A, Laser capture microdissection. Science, 1996. 274(5289): p. 998-1001.
19. Espina, V, Edmiston, K H, Heiby, M, Pierobon, M, Sciro, M, Merritt, B, Banks, S, Deng, J, VanMeter, A J, Geho, D H, Pastore, L, Sennesh, J, Petricoin, E F, 3rd, and Liotta, L A, A portrait of tissue phosphoprotein stability in the clinical tissue procurement process. Mol Cell Proteomics, 2008. 7(10): p. 1998-2018.
20. Ferrell, J E, Jr., Pomerening, J R, Kim, S Y, Trunnell, N B, Xiong, W, Huang, C Y, and Machleder, E M, Simple, realistic models of complex biological processes: positive feedback and bistability in a cell fate switch and a cell cycle oscillator. FEBS Lett, 2009. 583(24): p. 3999-4005.
21. Finocchiaro, L M, Arzt, E S, Fernandez-Castelo, S, Criscuolo, M, Finkielman, S, and Nahmod, V E, Serotonin and melatonin synthesis in peripheral blood mononuclear cells: stimulation by interferon-gamma as part of an immunomodulatory pathway. J Interferon Res, 1988. 8(6): p. 705-16.
22. Geho, D H, Petricoin, E F, Liotta, L A, and Araujo, R P, Modeling of protein signaling networks in clinical proteomics. Cold Spring Harb Symp Quant Biol, 2005. 70: p. 517-24.
23. Giannaccini, G, Betti, L, Palego, L, Schmid, L, Fabbrini, L, Pelosini, C, Gargini, C, Da Valle, Y, Lanza, M, Marsili, A, Maffei, M, Santini, F, Vitti, P, Pinchera, A, and Lucacchini, A, Human serotonin transporter expression during megakaryocytic differentiation of MEG-01 cells. Neurochem Res, 2010. 35(4): p. 628-35.
24. Goentoro, L, Shoval, O, Kirschner, M W, and Alon, U, The incoherent feedforward loop can provide fold-change detection in gene regulation. Mol Cell, 2009. 36(5): p. 894-9.
25. Hideshima, T, Mitsiades, C, Tonon, G, Richardson, P G, and Anderson, K C, Understanding multiple myeloma pathogenesis in the bone marrow to identify new therapeutic targets. Nat Rev Cancer, 2007. 7(8): p. 585-98.
26. Idzko, M, Panther, E, Stratz, C, Muller, T, Bayer, H, Zissel, G, Durk, T, Sorichter, S, Di Virgilio, F, Geissler, M, Fiebich, B, Herouy, Y, Elsner, P, Norgauer, J, and Ferrari, D, The serotoninergic receptors of human dendritic cells: identification and coupling to cytokine release. J Immunol, 2004. 172(10): p. 6011-9.
27. Jacquet, R, Hillyer, J, and Landis, W J, Analysis of connective tissues by laser capture microdissection and reverse transcriptase-polymerase chain reaction. Anal Biochem, 2005. 337(1): p. 22-34.
28. Kin, N W and Sanders, V M, It takes nerve to tell T and B cells what to do. J Leukoc Biol, 2006. 79(6): p. 1093-104.
29. Lago, F, Dieguez, C, Gomez-Reino, J, and Gualillo, 0, Adipokines as emerging mediators of immune response and inflammation. Nat Clin Pract Rheumatol, 2007. 3(12): p. 716-24.
30. Lauring, J, Abukhdeir, A M, Konishi, H, Garay, J P, Gustin, J P, Wang, Q, Arceci, R J, Matsui, W, and Park, B H, The multiple myeloma associated MMSET gene contributes to cellular adhesion, clonogenic growth, and tumorigenicity. Blood, 2008. 111(2): p. 856-64.
31. Matsui, W, Wang, Q, Barber, J P, Brennan, S, Smith, B D, Borrello, I, McNiece, I, Lin, L, Ambinder, R F, Peacock, C, Watkins, D N, Huff, C A, and Jones, R J, Clonogenic multiple myeloma progenitors, stem cell properties, and drug resistance. Cancer Res, 2008. 68(1): p. 190-7.
32. Mendez-Ferrer, S, Lucas, D, Battista, M, and Frenette, P S, Haematopoietic stem cell release is regulated by circadian oscillations. Nature, 2008. 452(7186): p. 442-7.
33. Mendez-Ferrer, S, Michurina, T V, Ferraro, F, Mazloom, A R, Macarthur, B D, Lira, S A, Scadden, D T, Ma'ayan, A, Enikolopov, G N, and Frenette, P S, Mesenchymal and haematopoietic stem cells form a unique bone marrow niche. Nature, 2010. 466(7308): p. 829-34.
34. Meredith, E J, Holder, M J, Chamba, A, Challa, A, Drake-Lee, A, Bunce, C M, Drayson, M T, Pilkington, G, Blakely, R D, Dyer, M J, Barnes, N M, and Gordon, J, The serotonin transporter (SLC6A4) is present in B-cell clones of diverse malignant origin: probing a potential anti-tumor target for psychotropics. FASEB J, 2005. 19(9): p. 1187-9.
35. Minges Wols, H A, Ippolito, J A, Yu, Z, Palmer, J L, White, F A, Le, P T, and Witte, P L, The effects of microenvironment and internal programming on plasma cell survival. Int Immunol, 2007. 19(7): p. 837-46.
36. Minges Wols, H A, Underhill, G H, Kansas, G S, and Witte, P L, The role of bone marrow-derived stromal cells in the maintenance of plasma cell longevity. J Immunol, 2002. 169(8): p. 4213-21.
37. Muller, T, Durk, T, Blumenthal, B, Grimm, M, Cicko, S, Panther, E, Sorichter, S, Herouy, Y, Di Virgilio, F, Ferrari, D, Norgauer, J, and Idzko, M, 5-hydroxytryptamine modulates migration, cytokine and chemokine release and T-cell priming capacity of dendritic cells in vitro and in vivo. PLoS One, 2009. 4(7): p. e6453.
38. Ossowska, K, Smialowska, M, Kuter, K, Wieronska, J, Zieba, B, Wardas, J, Nowak, P, Dabrowska, J, Bortel, A, Biedka, I, Schulze, G, and Rommelspacher, H, Degeneration of dopaminergic mesocortical neurons and activation of compensatory processes induced by a long-term paraquat administration in rats: implications for Parkinson's disease. Neuroscience, 2006. 141(4): p. 2155-65.
39. Park, E S, Shaughnessy, J D, Jr., Gupta, S, Wang, H, Lee, J S, Woo, H G, Zhan, F, Owens, J D, Jr., Potter, M, Janz, S, and Mushinski, J F, Gene expression profiling reveals different pathways related to Abl and other genes that cooperate with c-Myc in a model of plasma cell neoplasia. BMC Genomics, 2007. 8: p. 302.
40. Paweletz, C P, Charboneau, L, Bichsel, V E, Simone, N L, Chen, T, Gillespie, J W, Emmert-Buck, M R, Roth, M J, Petricoin, I E, and Liotta, L A, Reverse phase protein microarrays which capture disease progression show activation of pro-survival pathways at the cancer invasion front. Oncogene, 2001. 20(16): p. 1981-9.
41. Petricoin, E F, 3rd, Espina, V, Araujo, R P, Midura, B, Yeung, C, Wan, X, Eichler, G S, Johann, D J, Jr., Qualman, S, Tsokos, M, Krishnan, K, Helman, L J, and Liotta, L A, Phosphoprotein pathway mapping: Akt/mammalian target of rapamycin activation is negatively associated with childhood rhabdomyosarcoma survival. Cancer Res, 2007. 67(7): p. 3431-40.
42. Podojil, J R, Kin, N W, and Sanders, V M, CD86 and beta2-adrenergic receptor signaling pathways, respectively, increase Oct-2 and OCA-B Expression and binding to the 3′-IgH enhancer in B cells. J Biol Chem, 2004. 279(22): p. 23394-404.
43. Rinaldi, A, Chiaravalli, A M, Mian, M, Zucca, E, Tibiletti, M G, Capella, C, and Bertoni, F, Serotonin receptor 3A expression in normal and neoplastic B cells. Pathobiology, 2010. 77(3): p. 129-35.
44. Roato, I, Grano, M, Brunetti, G, Colucci, S, Mussa, A, Bertetto, 0, and Ferracini, R, Mechanisms of spontaneous osteoclastogenesis in cancer with bone involvement. FASEB J, 2005. 19(2): p. 228-30.
45. Russell, S T, Hirai, K, and Tisdale, M J, Role of beta3-adrenergic receptors in the action of a tumour lipid mobilizing factor. Br J Cancer, 2002. 86(3): p. 424-8.
46. Rutkowski, D T and Hegde, R S, Regulation of basal cellular physiology by the homeostatic unfolded protein response. J Cell Biol, 2010. 189(5): p. 783-94.
47. Shankavaram, U T, Reinhold, W C, Nishizuka, S, Major, S, Morita, D, Chary, K K, Reimers, M A, Scherf, U, Kahn, A, Dolginow, D, Cossman, J, Kaldjian, E P, Scudiero, D A, Petricoin, E, Liotta, L, Lee, J K, and Weinstein, J N, Transcript and protein expression profiles of the NCI-60 cancer cell panel: an integromic microarray study. Mol Cancer Ther, 2007. 6(3): p. 820-32.
48. Shaughnessy, J D, Jr., Haessler, J, van Rhee, F, Anaissie, E, Pineda-Roman, M, Cottler-Fox, M, Hollmig, K, Zangari, M, Mohiuddin, A, Alsayed, Y, Grazziutti, M, Epstein, J, Crowley, J, and Barlogie, B, Testing standard and genetic parameters in 220 patients with multiple myeloma with complete data sets: superiority of molecular genetics. Br J Haematol, 2007. 137(6): p. 530-6.
49. Shaughnessy, J D, Jr., Zhan, F, Burington, B E, Huang, Y, Colla, S, Hanamura, I, Stewart, J P, Kordsmeier, B, Randolph, C, Williams, D R, Xiao, Y, Xu, H, Epstein, J, Anaissie, E, Krishna, S G, Cottler-Fox, M, Hollmig, K, Mohiuddin, A, Pineda-Roman, M, Tricot, G, van Rhee, F, Sawyer, J, Alsayed, Y, Walker, R, Zangari, M, Crowley, J, and Barlogie, B, A validated gene expression model of high-risk multiple myeloma is defined by deregulated expression of genes mapping to chromosome 1. Blood, 2007. 109(6): p. 2276-84.
50. Sjogren, B and Svenningsson, P, Caveolin-1 affects serotonin binding and cell surface levels of human 5-HT7(a) receptors. FEBS Lett, 2007. 581(26): p. 5115-21.
51. Soga, F, Katoh, N, Inoue, T, and Kishimoto, S, Serotonin activates human monocytes and prevents apoptosis. J Invest Dermatol, 2007. 127(8): p. 1947-55.
52. Standal, T, Hjorth-Hansen, H, Rasmussen, T, Dahl, I M, Lenhoff, S, Brenne, A T, Seidel, C, Baykov, V, Waage, A, Borset, M, Sundan, A, and Hjertner, 0, Osteopontin is an adhesive factor for myeloma cells and is found in increased levels in plasma from patients with multiple myeloma. Haematologica, 2004. 89(2): p. 174-82.
53. Tonon, G, Molecular pathogenesis of multiple myeloma. Hematol Oncol Clin North Am, 2007. 21(6): p. 985-1006, vii.
54. Tyson, J J, Chen, K C, and Novak, B, Sniffers, buzzers, toggles and blinkers: dynamics of regulatory and signaling pathways in the cell. Curr Opin Cell Biol, 2003. 15(2): p. 221-31.
55. Underhill, G H, Minges Wols, H A, Fornek, J L, Witte, P L, and Kansas, G S, IgG plasma cells display a unique spectrum of leukocyte adhesion and homing molecules. Blood, 2002. 99(8): p. 2905-12.
56. VanMeter, A J, Rodriguez, A S, Bowman, E D, Jen, J, Harris, C C, Deng, J, Calvert, V S, Silvestri, A, Fredolini, C, Chandhoke, V, Petricoin, E F, 3rd, Liotta, L A, and Espina, V, Laser capture microdissection and protein microarray analysis of human non-small cell lung cancer: differential epidermal growth factor receptor (EGPR) phosphorylation events associated with mutated EGFR compared with wild type. Mol Cell Proteomics, 2008. 7(10): p. 1902-24.
57. Yadav, V K and Ducy, P, Lrp5 and bone formation: A serotonin-dependent pathway. Ann N Y Acad Sci, 2010. 1192(1): p. 103-9.
58. Yadav, V K, Oury, F, Suda, N, Liu, Z W, Gao, X B, Confavreux, C, Klemenhagen, K C, Tanaka, K F, Gingrich, J A, Guo, X E, Tecott, L H, Mann, J J, Hen, R, Horvath, T L, and Karsenty, G, A serotonin-dependent mechanism explains the leptin regulation of bone mass, appetite, and energy expenditure. Cell, 2009. 138(5): p. 976-89.
59. Yadav, V K, Ryu, J H, Suda, N, Tanaka, K F, Gingrich, J A, Schutz, G, Glorieux, F H, Chiang, C Y, Zajac, J D, Insogna, K L, Mann, J J, Hen, R, Ducy, P, and Karsenty, G, Lrp5 controls bone formation by inhibiting serotonin synthesis in the duodenum. Cell, 2008. 135(5): p. 825-37.
Yamazaki, K and Allen, T D, Ultrastructural morphometric study of efferent nerve terminals on murine bone marrow stromal cells, and the recognition of a novel anatomical unit: the “neuro-reticular complex”. Am J Anat, 1990. 187(3): p. 261-76.

What is claimed is:

1. A method of treating a mammalian patient diagnosed with bone disease comprising:
a. delivering to the tissues of said patient or administering to said patient a therapeutically effective amount of one or more compounds; and
b. wherein said one or more compounds comprises one or more of a tyrosine kinase inhibitor, a selective serotonin reuptake inhibitor (SSRI), a heterocyclic antidepressant, a monoamine oxidase inhibitor, an antidepressant, an anti-anxiety compound, an anti-epileptic, and an antibody.

2. The method of claim 1, wherein said bone disease comprises one or more of brittle bone disease, multiple myeloma, osteogenesis imperfecta (OI), osteolytic bone disease, amyloidosis, monoclonal gammopathy, alterations in bone marrow hematopoietic precursor cells, myelodysplasia, and bone metastasis.

3. The method of claim 1, wherein the monoamine oxidase inhibitor comprises one or more of a selective monoamine oxidase inhibitor, a monoamine oxidase A inhibitor, a monoamine oxidase B inhibitor or a nonselective monoamine oxidase inhibitor.

4. The method of claim 1, further comprising conjoint administration of a therapeutically effective amount of one or more therapeutic agents or therapeutic treatments selected from the group consisting of an autophagy inhibitor, a non-chemotherapeutic agent, a chemotherapeutic agent, an angiogenesis inhibitor, a bone breakdown inhibitor, an osteoclast or osteoblast activity inhibitor, and an immune signal modulator.

5. The method of claim 1, wherein serotonin is reduced in one or more of platelet cells, gastrointestinal cells, neural cells, immune cells, bone marrow microenvironment cells or cancer cells.

6. The method of claim 1, wherein said delivering comprises delivering said one or more compounds in an effective amount to modulate one or more of bone cell activity, stem cell activity, gastrointestinal cell activity, cancer cell activity, platelet cell activity, or neural cell activity.

7. The method of claim 1, wherein said mammalian patient is a human.

8. The method of claim 1, wherein said therapeutically effective amount of one or more compounds slows or arrests the growth of said bone disease.

9. The method of claim 1, wherein said one or more compounds are independently administered orally, topically, subcutaneously, parenterally, transdermally, mucosally, rectally, intranasally, via inhalation, via insufflation, via a patch, via application to the site of the tumor or tumor bed, via installation into a wound, by buccal, or by sublingual administration.

10. A method of detecting the presence of bone disease in a mammal comprising:
a. machine-assaying detectable serotonin in a biological sample of said mammal;
b. comparing the detectable serotonin in said biological sample with a positive or negative control; and
c. identifying the presence of bone disease based on said detectable serotonin.

11. The method of claim 10, wherein said bone disease comprises one or more of brittle bone disease, multiple myeloma, osteogenesis imperfecta (OI), osteolytic bone disease, amyloidosis, monoclonal gammopathy, alterations in bone marrow hematopoietic precursor cells, myelodysplasia, and bone metastasis.

12. The method of claim 10, wherein said biological sample comprises one or more of isolated bone marrow aspirate, isolated bone tissue, isolated blood plasma, isolated blood serum, isolated blood cells, and isolated whole blood.

13. The method of claim 10, further comprising treating said mammal based on said identification of the presence of bone disease.

14. The method of claim 10, further comprising regulating bone remodeling in the subject including modulating serotonin in the subject based on said detecting.

15. The method of claim 10, wherein said assaying comprises at least one of:
a. post translational modification of signaling proteins;
b. caspase cleavage;
c. poly(ADP-ribose) polymerase (PARP) cleavage or dye exclusion/uptake;
d. reverse phase microarray (RPMA);
e. ELISA;
f. flow cytometry;
g. Immunohistochemistry;
h. Immunoassay;
i. high resolution mass spectroscopy;
j. suspension bead array; and
k. Western blot.

16. A method of treating bone disease, in a subject, comprising:
a. administering an autophagy pathway inhibitor; and
b. administering one or more of a serotonin modulator, a tyrosine kinase inhibitor, an antidepressant, an anti-anxiety compound, an antiepileptic, a monoamine oxidase inhibitor, an antibody, a non-chemotherapeutic agent or a bisphosphonate; and
c. wherein the bone disease may be one or more of monoclonal gammopathy of unknown significance (MGUS), premalignant bone marrow cells and multiple myeloma.

17. The method of claim 16, wherein said autophagy pathway inhibitor comprises a 4-Amino-quinoline.

18. The method of claim 16, wherein said autophagy pathway inhibitor comprises a 4-Amino-2-Alkyl-quinoline, a 2-Methyl-quinoline, a 4-Amino-3-Bromo-quinoline, a 4-Amino-3-Chloro-quinoline, a 4-Amino-6-Bromo-quinoline, a 4-Amino-7-Bromo-quinoline, or a 4-Amino-8-Bromo-quinoline.

19. The method of claim 16, wherein said bisphosphonate comprises one or more of alendronate, pamidronate or zoledronic acid.

20. The method of claim 16, wherein said autophagy pathway inhibitor comprises chloroquine.
